(19) United States
(12) Patent Application Publication
(10) Pub. No.: US 2004/0242918 A1
(43) Pub. Date: Dec. 2, 2004

(54) Support and catalyst for use in producing lower aliphatic carboxylic acid ester, process for producing the catalyst and process for producing lower aliphatic carboxylic acid ester using the catalyst

(75) Inventors: Etsuko Kadowaki, Oita-shi (JP); Tomoyoshi Higashi, Oita-shi (JP); Wataru Oguchi, Oita-shi (JP); Hiroshi Uchida, Oita-shi (JP); Kousuke Narumi, Oita-shi (JP)

Correspondence Address: SUGHRUE MION, PLLC, 2100 PENNSYLVANIA AVENUE, N.W., SUITE 800, WASHINGTON, DC, 20037, US

(73) Assignee: SHOWA DENKO K.K

(21) Appl. No.: 10/785,229

(22) Filed: Feb. 25, 2004

Related U.S. Application Data
(63) Continuation-in-part of application No. 09/889,354, filed on Jul. 17, 2001, now abandoned.
Application No. 09/889,354 is a 371 of international application No. PCT/JP01/05532, filed on Jun. 27, 2001.
(63) Continuation-in-part of application No. 10/070,259, filed on Mar. 4, 2002, now abandoned.
Application No. 10/070,259 is a 371 of international application No. PCT/JP02/01156, filed on Feb. 12, 2002.
(60) Provisional application No. 60/218,803, filed on Jul. 18, 2000.
(60) Provisional application No. 60/273,343, filed on Mar. 6, 2001.

(30) Foreign Application Priority Data
Jun. 27, 2000 (JP) 2000-192964(PAT.
Feb. 13, 2001 (JP) 2001-036038(PAT.
Dec. 7, 2001 (JP) 2001-373675(PAT.

(51) Int. Cl. C07C 67/02; B01J 27/19

(52) U.S. Cl. 560/241; 502/211

(57) ABSTRACT

A catalyst for use in producing a lower aliphatic carboxylic acid ester, wherein the catalyst is produced by a process comprising a step of contacting the catalyst with a gas containing at least one member selected from water, lower aliphatic carboxylic acids and lower aliphatic alcohols; a process for producing the catalyst; and a process for producing a lower aliphatic carboxylic acid ester using the catalyst. The catalyst can exhibit high initial activity and high space time yield, ensure sufficiently long catalyst life in practice in industry, and can prevent the production of by-product materials. A siliceous support for use in a catalyst, which has a silicon content of from 39.7 to 46.3% by mass or a silicon content of from 85 to 99% by mass in terms of silicon dioxide or a crush strength of 30 N or more. By the use of a catalyst comprising the support, a lower aliphatic carboxylic acid ester is produced from lower olefin and a lower aliphatic carboxylic acid in a gas phase without causing great reduction of catalytic activity or cracking or abrasion of the catalyst.

### CROSS-REFERENCE TO RELATED APPLICATIONS

This application is a continuation-in-part application of application Ser. No. 09/889,354 filed Jul. 17, 2001, which was the National Stage of PCT/JP01/05532 filed Jun. 27, 2001, which claims benefit pursuant to 35 U.S.C. § 119(e)(1) of the filing date of the Provisional Application No. 60/218,803 filed Jul. 18, 2000, pursuant to 35 U.S.C. § 111(b), and is a continuation-in-part application of application Ser. No. 10/070,259 filed Mar. 4, 2002, which was the National Stage of PCT/JP02/01156 filed Feb. 12, 2002, which claims benefit of the Provisional Application No. 60/273,343 filed on Mar. 6, 2001, the disclosures of all of which are incorporated herein by reference.

### TECHNICAL FIELD

The present invention relates to a catalyst for use in producing a lower aliphatic carboxylic acid ester from a lower olefin and a lower aliphatic carboxylic acid, a process for producing the catalyst, and a process for producing a lower aliphatic carboxylic acid ester using the catalyst.

More specifically, the present invention relates to a catalyst for use in producing a lower aliphatic carboxylic acid ester, which contains a specific heteropolyacid salt and which is used in the process for producing a lower aliphatic carboxylic acid ester by esterifying a lower aliphatic carboxylic acid with a lower olefin, wherein the catalyst is contacted with a gas containing at least one member selected from the group consisting of water, lower aliphatic carboxylic acids and lower aliphatic alcohols before the reaction; a process for producing the catalyst; and a process for producing a lower aliphatic carboxylic acid ester using the catalyst.

The present invention also relates to a support for use in a catalyst for producing a lower aliphatic carboxylic acid ester; a catalyst for producing a lower aliphatic carboxylic acid ester using the support; a process for producing the catalyst; and a process for producing a lower aliphatic carboxylic acid ester using the catalyst. More specifically, the present invention relates to a siliceous support for use in a catalyst for producing a lower aliphatic carboxylic acid ester from a lower olefin and a lower aliphatic carboxylic acid; a catalyst for producing a lower aliphatic carboxylic acid ester using the support; a process for producing the catalyst; and a process for producing a lower aliphatic carboxylic acid ester using the catalyst.

### BACKGROUND ART

It is well known that a corresponding ester can be produced from a lower aliphatic carboxylic acid and an olefin. Also, a catalyst comprising a heteropolyacid and/or a salt thereof is known to effectively act in this reaction. Specific examples of such a catalyst include those described in Japanese Unexamined Patent Publications No. 4-139148 (JP-A-4-139148), No. 4-13949 (JP-A-4-139149), No. 5-65248 (JP-A-5-65248) and No. 5-29489 (JP-A-5-294894).

Among these specific examples, the catalysts containing at least one salt selected from the group consisting of cesium salts, rubidium salts, thallium salts, ammonium salts and potassium salts of phosphotungstic acid, silicotungstic acid, phosphomolybdic acid and silicomolybdic acid disclosed in JP-A-4-139148, JP-A-4-139149 and JP-A-5-65248, exhibit high initial activity and relatively high space time yield. However, in use on an industrial base, for example, the activity decreases and the space time yield also lowers with the progress of reaction and this was a problem to be solved.

In order to solve this problem, Japanese Unexamined Patent Publication No. 5-170698 (JP-A-5-170698) proposes a method of contacting a catalyst described above with water and an organic carboxylic acid or an organic carboxylic acid ester in a gas phase before performing the reaction. In a production process of a lower aliphatic carboxylic acid ester using a catalyst subjected to a pre-contacting treatment according to the above-described method, it is revealed that the catalyst exhibits high initial activity and the activity scarcely decreases as compared with conventional production processes using a catalyst which is not subjected to the contacting treatment.

On the other hand, Japanese Unexamined Patent Publication No. 5-294894 (JP-A-5-294894) discloses a catalyst obtained by loading at least one heteropolyacid salt selected from the group consisting of lithium salts, copper salts, magnesium salts and gallium salts of heteropolyacids on a support and it is revealed that although the method disclosed in JP-A-5-170698 of contacting a catalyst with water and an organic carboxylic acid or an organic carboxylic acid ester in a gas phase before performing the reaction is not used, the catalyst can exhibit initial activity equal thereto or higher than that and can maintain the activity.

In other words, with respect to the initial activity and space time yield of the catalyst and the maintenance thereof (so-called catalyst life), the problems have been overcome by the catalyst disclosed in JP-A-5-294894. The matter in need of improvement is the production of by-products such as olefins, having 3 or more carbon atoms including butene, and aldehydes.

The production of these by-products directly causes reduction in the selectivity of reaction. Furthermore, these by-products sometimes become catalyst poisons in the reaction of a lower olefin with a lower aliphatic carboxylic acid and may conspicuously inhibit the reaction.

Particularly, in industrially performing the process for producing a lower alphatic carboxylic acid ester through the reaction, a circulation process is generally employed mainly for recycling unreacted raw materials so as to increase the reaction efficiency in view of profitability. In this case, if those by-products are not removed and returned to a reaction system via a circulation system, the catalyst may be damaged and the catalyst life greatly shortened.

To solve this problem, the methods described, for example, in Japanese Unexamined Patent Publications No. 11-269126 (JP-A-11-269126) and No. 11-335323 (JP-A-11-335323) may be used. JP-A-11-269126 discloses a method of removing acetaldehyde impurities at the reactor inlet by distillation or by reacting them with a compound highly reactive with an aldehyde group. Also, JP-A-11-335323 discloses a method of newly providing a step for removing acetaldehyde using an acetaldehyde-removing column and thereby separating it. In either case, however, a new separation step must be provided, therefore, the process becomes very cumbersome and the costs increase.

As such, it has been proposed to provide a step for separating and removing by-products so that the catalyst life can be maintained even in the process employing a circulation system. However, means to solve this problem from the standpoint of sufficiently suppressing the production of by-products themselves in the reaction has not yet been found.

As is well known, a corresponding ester can be produced from a lower aliphatic carboxylic acid and an olefin by a gas phase catalytic reaction. Also, a catalyst comprising a heteropolyacid and/or a heteropolyacid salt and supported on a siliceous support is known to be useful in such a reaction. The siliceous support used here is known as a silica support. Specific examples of recent publications disclosing this technique include Japanese Unexamined Patent Publication No. 11-269126 (JP-A-11-269126) and Japanese Unexamined Patent Publication No. 11-263748 (JP-A-11-263748).

JP-A-11-269126 and JP-A-11-263748 disclose a technique of producing a lower aliphatic carboxylic acid ester by contacting a lower aliphatic carboxylic acid and a lower olefin with a heteropolyacid supported on a silica support (siliceous support) in a gas phase. In these patent publications, it is stated that, to achieve optimal performance, the silica support preferably has a purity of 99% by weight or more, because impurities may adversely affect the catalytic activity. As such, the siliceous support used as a support for improving the catalytic activity preferably has a high silicon purity. However, siliceous supports having a high silicon purity suffer from a very low strength and if a catalyst using such a support having a low strength is used, cracking may be generated at the preparation of the catalyst, or cracking or abrasion of the catalyst may be generated according to the amount of use in the production of esters, giving rise to an increase in the pressure loss of a reactor and, in turn, to failure of safe operation.

### DISCLOSURE OF INVENTION

An object of the present invention is to provide a catalyst for use in producing a lower aliphatic carboxylic acid ester, which is used in producing a lower aliphatic carboxylic acid ester from a lower olefin and a lower aliphatic carboxylic acid and which is a catalyst having high initial activity and high space time yield, favored with a catalyst life long enough to endure practical use in industry and capable of reducing the production of by-products; a process for producing the catalyst; and a process for producing a lower aliphatic carboxylic acid ester using the catalyst.

The present inventors have made extensive investigations into a catalyst not only capable of exhibiting high initial activity and sufficiently long catalyst life at the reaction of a lower olefin and a lower aliphatic carboxylic acid in a gas phase to produce a lower fatty acid ester, but also reduced in the production of by-products represented by butene and aldehydes which work out to a catalyst poison. As a result, it has been found that when a specific step is provided in the preparation of a catalyst, the catalyst can be greatly reduced in the production of by-products harmful to the catalyst, such as butene and aldehydes. The present invention has been accomplished based on this finding.

More specifically, the present invention (I) is a catalyst for use in producing a lower aliphatic carboxylic acid ester, which is used in reacting a lower olefin with a lower aliphatic carboxylic acid in a gas phase to produce a lower aliphatic carboxylic acid ester, wherein the catalyst is produced by a process comprising the following first and second steps:

First Step

a step for loading one or more heteropolyacid salts on a support to obtain a heteropolyacid salt supported catalyst; and

Second Step

a step for contacting the heteropolyacid salt supported catalyst obtained in the first step with a gas containing at least one member selected from the group consisting of water, lower aliphatic carboxylic acids and lower aliphatic alcohols to obtain the catalyst for use in producing a lower aliphatic carboxylic acid ester.

The second step generally is conducted in the absence of any lower olefin and thus is conducted before feeding any lower olefin.

The present invention (II) is a process for producing a catalyst for use in producing a lower aliphatic carboxylic acid ester, the catalyst being used in reacting a lower olefin with a lower aliphatic carboxylic acid in a gas phase to produce a lower aliphatic carboxylic acid ester, which process comprises the following first and second steps:

First Step

a step for loading one or more heteropolyacid salts on a support to obtain a heteropolyacid salt supported catalyst; and

Second Step

a step for contacting the heteropolyacid salt supported catalyst obtained in the first step with a gas containing at least one member selected from the group consisting of water, lower aliphatic carboxylic acids and lower aliphatic alcohols to obtain the catalyst for use in producing a lower aliphatic carboxylic acid ester.

The second step generally is conducted in the absence of any lower olefin and thus is conducted before feeding any lower olefin.

The present invention (III) is a process for producing a lower aliphatic carboxylic acid ester, comprising reacting a lower olefin with a lower aliphatic carboxylic acid in a gas phase in the presence of the catalyst for use in producing a lower aliphatic carboxylic acid ester of the present invention (I).

The present invention (IV) is a process for producing a lower aliphatic carboxylic acid ester comprising reacting a lower olefin with a lower aliphatic carboxylic acid in a gas phase, which process comprises the following first to fourth steps:

First Step

a step for loading one or more heteropolyacid salts on a support to obtain a heteropolyacid salt supported catalyst;

Second Step

a step of filling the heteropolyacid salt supported catalyst obtained in the first step, into a reactor for use in the reaction of the lower olefin with the lower aliphatic carboxylic acid in a gas phase;

Third Step

a step of contacting the heteropolyacid salt supported catalyst filled in the reactor, with a gas containing at least one member selected from the group consisting of water, lower aliphatic carboxylic acids and lower aliphatic alcohols; and

Fourth Step

a step of passing a mixed gas containing the lower olefin and the lower aliphatic carboxylic acid through the heteropolyacid salt supported catalyst after the third step, to obtain the lower aliphatic carboxylic acid ester.

The third step generally is conducted in the absence of any lower olefin and thus is conducted before feeding any lower olefin.

In another aspect, an object of the present invention is to provide a support capable of providing a stably operable catalyst, for producing a lower aliphatic carboxylic acid ester, which prevents a great reduction in the catalytic activity and protects the catalyst from cracking or abrasion during the production of a lower aliphatic carboxylic acid ester, wherein the catalyst is supported on a siliceous support and used in the production of a lower aliphatic carboxylic acid ester from a lower olefin and a lower aliphatic carboxylic acid. In this aspect, the object of the present invention includes providing a catalyst for producing a lower aliphatic carboxylic acid ester using the support, a process for producing the catalyst and a process for producing a lower aliphatic carboxylic acid ester using the catalyst.

To attain the above-described objects of this aspect, the present invention (V) provides a siliceous support for use in a catalyst for producing a lower aliphatic carboxylic acid ester by reacting a lower olefin with a lower aliphatic carboxylic acid in a gas phase, which has a silicon content of from 39.7 to 46.3% by mass.

The present invention (VI) also provides a siliceous support for use in a catalyst for producing a lower aliphatic carboxylic acid ester by reacting a lower olefin with a lower aliphatic carboxylic acid in a gas phase, which has a silicon content of from 85 to 99% by mass in terms of silicon dioxide.

The present invention (VII) also provides a siliceous support for use in a catalyst for producing a lower aliphatic carboxylic acid ester by reacting a lower olefin with a lower aliphatic carboxylic acid in a gas phase, which has a crush strength of 30 N or more.

The present invention (VIII) also provides a catalyst supported on a support, which is a catalyst for producing a lower aliphatic carboxylic acid ester by reacting a lower olefin with a lower aliphatic carboxylic acid in a gas phase, wherein the support is any one of the above-described supports of the present invention.

The present invention (IX) also provides a process for producing a catalyst for producing a lower aliphatic carboxylic acid ester by reacting a lower olefin with a lower aliphatic carboxylic acid in a gas phase, the process comprising a step of loading at least one member selected from the group consisting of heteropolyacids and salts thereof on any one of the above-described supports of the present invention.

The present invention (X) also provides a process for producing a catalyst for producing a lower aliphatic carboxylic acid ester by reacting a lower olefin with a lower aliphatic carboxylic acid in a gas phase, the process comprising the following first and second steps:

First Step:

a step of loading at least one member selected from the group consisting of heteropolyacids and salts thereof on any one of the above-described supports of the present invention to obtain a heteropolyacid and/or heteropolyacid salt-supported catalyst;

Second Step:

a step of contacting the heteropolyacid and/or heteropolyacid salt-supported catalyst obtained in the first step with a gas containing at least one member selected from the group consisting of water, lower aliphatic carboxylic acids and lower aliphatic alcohols to obtain a catalyst for producing a lower aliphatic carboxylic acid ester.

The present invention (XI) also provides a process for producing a lower aliphatic carboxylic acid ester, comprising reacting a lower olefin with a lower aliphatic carboxylic acid in a gas phase in the presence of the above-described catalyst (VIII) for producing a lower aliphatic carboxylic acid ester of the present invention or the catalysts obtained in the above described inventions (IX) and (X).

### BRIEF DESCRIPTION OF DRAWINGS

FIG. 1 is a graph showing the crush strength and the specific activity with respect to the silica content of the support in catalysts used in certain Examples of the present invention and Comparative Examples.

### DETAILED DESCRIPTION OF THE INVENTION

The present invention will be described below with reference to preferred embodiments thereof.

### A. The Inventions (I) to (IV)

The present invention (I) is a catalyst for use in producing a lower aliphatic carboxylic acid ester, which is used in reacting a lower olefin with a lower aliphatic carboxylic acid in a gas phase to produce a lower aliphatic carboxylic acid ester, wherein the catalyst is produced by a process comprising the following first and second steps:

First Step

a step for loading one or more heteropolyacid salts on a support to obtain a heteropolyacid salt supported catalyst; and

Second Step

a step for contacting the heteropolyacid salt supported catalyst obtained in the first step with a gas containing at least one member selected from the group consisting of water, lower aliphatic carboxylic acids and lower aliphatic alcohols to obtain the catalyst for use in producing a lower aliphatic carboxylic acid ester.

That is, the catalyst for use in producing a lower aliphatic carboxylic acid ester of the present invention (I) is a catalyst obtained by a production process comprising the above-described first and second steps.

The first step is described below.

In the catalyst for use in producing a lower aliphatic carboxylic acid ester of the present invention (I), the heteropolyacid used in the first step comprises a center element and a peripheral element to which oxygen is bonded. The center element is usually silicon or phosphorus but may comprise an optional element selected from various kinds of elements belonging to Groups 1 to 17 of the Periodic Table.

Specific examples of the center element include cupric ion; divalent beryllium, zinc, cobalt and nickel ions; trivalent boron, aluminum, gallium, iron, cerium, arsenic, antimony, phosphorus, bismuth, chromium and rhodium ions; tetravalent silicon, germanium, tin, titanium, zirconium, vanadium, sulfur, tellurium, manganese, nickel, platinum, thorium, hafnium and cerium ions and other rare earth ions; pentavalent phosphorus, arsenic, vanadium and antimony ions; hexavalent tellurium ion; and heptavalent iodide ion, however, the present invention is by no means limited thereto. Specific examples of the peripheral element include tungsten, molybdenum, vanadium, niobium and tantalum, however, the present invention is by no means limited thereto.

These heteropolyacids are also known as a “polyoxo-anion”, a “polyoxometallic salt” or a “metal oxide cluster”. Some well known structures of anions are named after a researcher himself in this field, for example, as Keggin, Wells-Dawson or Anderson-Evans-Perloff structures. There are detailed description in “Porisan no Kagaku, Kikan Kagaku Sousetau (Chemistry of Polyacids, Seasonal Publication for Introduction to Chemistry)”, No. 20, 1993, edited by Japan Chemical Society. Heteropolyacids usually have a high molecular weight, for example, a molecular weight of 700 to 8,500, and include dimeric complexes.

Specific examples of the heteropolyacid which can be used as the starting material of heteropolyacid salt in the catalyst for use in producing a lower aliphatic carboxylic acid ester of the present invention (I) include:

| | |
|---|---|
| Tungstosilicic acid | H₄[SiW₁₂O₄₀].xH₂O |
| Tungstophosphoric acid | H₃[PW₁₂O₄₀].xH₂O |
| Molybdophosphoric acid | H₃[PMO₁₂O₄₀]xH₂O |
| Molybdosilicic acid | H₄[SiMo₁₂O₄₀].xH₂O |
| Vanadotungstosilicic acid | H₄₊ₙ[SiVₙW₁₂₋ₙO₄₀].xH₂O |
| Vanadotungstophosphoric acid | H₃₊ₙ[PVₙW₁₂₋ₙO₄₀].Xh₂O |
| Vanadomolybdophosphoric acid | H₃₊ₙ[PVₙMo₁₂₋ₙO₄₀].xH₂O |
| Vanadomolybdosilicic acid | H₄₊ₙ[SiVₙMO₁₂₋ₙO₄₀].xH₂O |
| Molybdotungstosilicic acid | H₄[SiMOₙW₁₂₋ₙO₄₀].xH₂O |
| Molybdotungstophosphoric acid | H₃[PMOₙW₁₂₋ₙO₄₀].xH₂O |

wherein n is an integer of 1 to 11 and x is an integer of at least 1. However, the present invention is by no means limited thereto.

Among these, preferred are tungstosilicic acid, tungstophosphoric acid, molybdophosphoric acid, molybdo-silicic acid, vanadotungstosilicic acid and vanadotungstophosphoric acid, and more preferred are tungstosilicic acid, tungstophosphoric acid, vanadotungstosilicic acid and vanadotungstophosphoric acid.

The method of the production of the heteropolyacids are not particularly limited and any method can be used. For example, they may be produced by heating an acidic aqueous solution (a pH of about 1-2) containing a salt of molybdec acid or tungstic acid and a simple oxygen acid of a hetero-atom or a salt thereof. A heteropolyacid compound may be isolated from the resulting aqueous heteropolyacid solution by a method of separation through precipitation as a metal salt, for example. Specific examples are described in “Shin Jikken Kagaku Kouza 8, Muki Kagoubutsu no Gousei (III) (New Experimental Chemistry Course 8, Synthesis of Inorganic Compounds)”, edited by Japan Chemical Society, published by Maruzen K.K., Aug. 20, 1984, Third Edition, page 1413, but the present invention is by no means limited thereto. The Keggin structure of the obtained heteropolyacid can be confirmed by chemical analysis as well as X-ray diffraction, UV and IR measurements.

The heteropolyacid salt as used in the catalyst for use in producing a lower aliphatic carboxylic acid ester of the present invention (I) is not particularly limited as long as it is a metal salt or an onium salt in which a part or all of hydrogen atoms of the above-described heteropolyacids are substituted. Specific examples thereof include salts of metals such as lithium, sodium, magnesium, barium, copper, gold and gallium, and onium salts, however, the present invention is not limited thereto. Among these, preferred are lithium salts, sodium salts, gallium salts, copper salts and gold salts, more preferred are lithium salts, sodium salts and copper salts.

Heteropolyacids have a relatively high solubility in a polar solvent such as water and other oxygen-containing solvents particularly when the heteropolyacids are free acids or comprise several salts, and the solubility can be controlled by appropriately selecting the counter ion.

Examples of the starting material for the metal element or onium moiety of forming the heteropolyacid salt in the present invention include lithium nitrate, lithium acetate, lithium sulfate, lithium sulfite, lithium carbonate, lithium phosphate, lithium oxalate, lithium nitrite, lithium chloride, lithium citrate, sodium nitrate, sodium acetate, sodium sulfate, sodium carbonate, monosodium phosphate, disodium phosphate, sodium oxalate, sodium nitrite, sodium chloride, sodium citrate, magnesium nitrate hexahydrate, magnesium acetate tetrahydrate, magnesium sulfate, magnesium carbonate, magnesium phosphate tricosahydrate, magnesium oxalate dihydrate, magnesium chloride, magnesium citrate, barium nitrate, barium acetate, barium sulfate, barium carbonate, barium hydrogenphosphate, barium oxalate monohydrate, barium sulfite, barium chloride, barium citrate, copper nitrate, copper acetate, copper sulfate, copper carbonate, copper diphosphate, copper oxalate, copper chloride, copper citrate, aurous chloride, chloroauric acid, auric oxide, auric hydroxide, auric sulfide, aurous sulfide, gallium dichloride, gallium monochloride, gallium citrate, gallium acetate, gallium nitrate, gallium sulfate, gallium phosphate, ammonium acetate, ammonium carbonate, ammonium nitrate, ammonium dihydrogenphosphate, ammonium hydrogencarbonate, ammonium citrate, ammonium nitrate, diammonium phosphate, monoammonium phosphate and ammonium sulfate, however, the present invention is by no means limited thereto.

Among these, preferred are lithium nitrate, lithium acetate, lithium carbonate, lithium oxalate, lithium citrate, sodium nitrate, sodium acetate, sodium carbonate, sodium oxalate, sodium citrate, copper nitrate, copper acetate, copper carbonate, copper citrate, aurous chloride, chloroauric acid, gallium citrate, gallium acetate and gallium nitrate, and more preferred are lithium nitrate, lithium acetate, lithium carbonate, lithium oxalate, lithium citrate, sodium nitrate, sodium acetate, sodium carbonate, sodium oxalate, sodium citrate, copper nitrate, copper acetate, copper carbonate and copper citrate.

Specific examples of the heteropolyacid salt which can be used in the catalyst for use in producing a lower aliphatic carboxylic acid ester of the present invention (I) include lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of molybdophosphoric acid, sodium salt of molybdophosphoric acid, copper salt of molybdophosphoric acid, gold salt of molybdophosphoric acid, gallium salt of molybdophosphoric acid, lithium salt of molybdosilicic acid, sodium salt of molybdosilicic acid, copper salt of molybdosilicic acid, gold salt of molybdosilicic acid, gallium salt of molybdosilicic acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid, gallium salt of vanadotungstophosphoric acid, lithium salt of vanadomolybdophosphoric acid, sodium salt of vanadomolybdophosphoric acid, copper salt of vanadomolybdophosphoric acid, gold salt of vanadomolybdophosphoric acid, gallium salt of vanadomolybdophosphoric acid, lithium salt of vanadomolybdosilicic acid, sodium salt of vanadomolybdosilicic acid, copper salt of vanadomolybdosilicic acid, gold salt of vanadomolybdosilicic acid and gallium salt of vanadomolybdosilicic acid.

Among these, preferred are lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of molybdophosphoric acid, sodium salt of molybdophosphoric acid, copper salt of molybdophosphoric acid, gold salt of molybdophosphoric acid, gallium salt of molybdophosphoric acid, lithium salt of molybdosilicic acid, sodium salt of molybdosilicic acid, copper salt of molybdosilicic acid, gold salt of molybdosilicic acid, gallium salt of molybdosilicic acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid and gallium salt of vanadotungstophosphoric acid.

More preferred are lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid and gallium salt of vanadotungstophosphoric acid.

In the catalyst for use in producing a lower aliphatic carboxylic acid ester of the present invention (I), the heteropolyacid salt as a catalytic activity component is supported on a support. Substances which can be used as the support are not particularly limited and porous substances commonly used as a support may be used. Specific examples thereof include those comprising silica, diatomaceous earth, montmorillonite, titania, activated carbon, alumina and silica alumina, preferably silica, silica alumina and montmorillonite.

The support is also not limited on the shape thereof and may be in the powder, spherical, pellet-like or any other form. A sphere or pellet-like form is preferred. Furthermore, the particle size is not particularly limited and although the preferred particle size varies depending on the reaction form, the average diameter is preferably from 2 to 10 mm in the case of use in a fixed bed system and from powder to 5 mm in the case of use in a fluidized bed system.

The support is most preferably a spherical or pellet-form siliceous support.

The method for loading the heteropolyacid salt on the support in the first step of the catalyst for producing a lower aliphatic carboxylic acid ester of the present invention (I) is roughly classified into the following three groups (1) to (3).

(1) A method of loading a desired heteropolyacid on a support and thereafter loading a starting material for the desired element or moiety of forming the salt.

(2) A method of loading a desired heteropolyacid together with a starting material for the element or moiety of forming the salt or loading a previously prepared heteropolyacid salt on a support.

(3) A method of previously loading a starting material for the element or moiety of forming the salt on a support and thereafter loading a desired heteropolyacid thereon.

In any of these methods (1) to (3), the heteropolyacid and the starting material for the element or moiety of forming the salt each can be loaded by dissolving or suspending it in an appropriate solvent. The solvent may be any as long as it can uniformly dissolve or suspend the desired heteropolyacid, a salt thereof and the starting material for the element or moiety of forming the salt, and examples of the solvent which can be used include water, an organic solvent and a mixture thereof. Among these, preferred are water, alcohols and carboxylic acids.

The method used for the dissolution or suspension may also be any as long as it can uniformly dissolve or suspend the desired heteropolyacid, a salt thereof and the starting material for the element or moiety of forming the salt. In the case of a free acid, a free acid which can dissolve may be dissolved as it is in a solvent and even in the case of a free acid which cannot completely dissolve, if the free acid can be uniformly suspended by forming it into fine powder, the free acid may be suspended as such.

In the method (1), a solution or suspension obtained by dissolving or suspending a heteropolyacid in a solvent is absorbed to a support to thereby load the heteropolyacid on the support and, then, a solution or suspension of a starting material for the element or moiety of forming a desired salt is absorbed to the support to thereby load the element or moiety. At this time, a neutralization reaction proceeds on the support, as a result, a heteropolyacid salt supported catalyst can be prepared.

In the method (2), a heteropolyacid and a starting material for the element or moiety of forming the salt are dissolved or suspended together or separately and then mixed to prepare a uniform solution or suspension, and the solution or suspension is absorbed to a support, thereby loading the heteropolyacid and the element or moiety. In the case of a compound in the state of a heteropolyacid salt, a uniform solution or suspension may be obtained in the same manner as in the case of a free acid.

In the method (3), a solution or suspension of a starting material for the element or moiety of forming the salt is previously prepared, the solution or suspension is absorbed to a support to thereby load the element or moiety, and then a desired heteropolyacid is loaded. This method includes a method of using an element or moiety which is previously contained in the support and which can form a heteropolyacid salt.

More specifically, a part or all of the elements previously contained in a support sometimes act to form a salt of a heteropolyacid when the heteropolyacid is loaded, and as a result, a heteropolyacid salt is formed. Examples of such an element include potassium, sodium, calcium, iron, magnesium, titanium and aluminum, however, the present invention is not limited thereto.

The kind of the element previously contained in a support and the amount thereof can be determined by a chemical analysis such as inductively coupled plasma emission spectrometry (hereinafter referred to as “ICP”), fluorescent X-ray method and atomic absorption method. The kind and the amount of the element vary depending on the support, however, potassium, sodium, calcium, iron, magnesium, titanium and aluminum are sometimes contained in a relatively large amount and the content thereof is approximately from 0.001 to 5.0% by mass. Therefore, depending on the combination of a support and a heteropolyacid, the element previously contained in the support may be in an amount large enough to form a salt, though this may vary depending on the kind and the amount of the heteropolyacid to be supported.

The method for loading a solution or suspension of heteropolyacid or a salt thereof on a support is not particularly limited and a known method may be used. Specifically, for example, the catalyst may be prepared by dissolving a heteropolyacid in a distilled water corresponding to the liquid absorption amount of the support used and impregnating the solution into a support. Also, the catalyst may be prepared using an excess aqueous heteropolyacid solution by impregnating it into a support while appropriately moving the support in the heteropolyacid solution and then removing the excess acid by filtration. The volume of the solution or suspension used at this time varies depending on the support used or the loading method thereon.

The thus-obtained wet catalyst is suitably dried by placing it in a heating oven for a few hours. The drying method is not particularly limited and any method such as of a standing or belt conveyor system may be used. After drying, the catalyst is cooled to the ambient temperature in a desiccator so as not to absorb moisture.

The amount of the heteropolyacid salt supported in the heteropolyacid salt supported catalyst can be simply calculated by subtracting the weight of the support used from the weight after drying of the catalyst prepared. The amount supported may be more exactly determined by a chemical analysis such as ICP, fluorescent x-ray method or atomic absorption method.

The amount of the heteropolyacid salt supported is preferably from 10 to 150% by mass, more preferably from 30 to 100% by mass, based on the entire weight of the support.

If the heteropolyacid salt content is less than 10% by mass, the content of active components in the catalyst is too small and the activity per the catalyst unit weight may disadvantageously decrease. If the heteropolyacid salt content exceeds 150% by mass, the effective pore volume may decrease, as a result, the effect owing to the increase in the supported amount may not be brought out and at the same time, coking may be disadvantageously liable to occur to greatly shorten the catalyst life.

The second step is described below.

The second step in the catalyst for use in producing a lower aliphatic carboxylic acid ester of the present invention (I) is a step for contacting the heteropolyacid salt supported catalyst obtained in the first step with a gas containing at least one member selected from the group consisting of water, lower aliphatic carboxylic acids and lower aliphatic alcohols.

The term “contacting” as used above refers to bringing the heteropolyacid salt supported catalyst obtained in the first step into contact with a gas containing at least one member selected from the group consisting of water, lower aliphatic carboxylic acids and lower aliphatic alcohols. The method for the contacting is not particularly limited and, for example, the following methods may be used.

(a) A method of placing the heteropolyacid salt supported catalyst obtained in the first step in an atmosphere of a gas containing at least one member selected from the group consisting of water, lower aliphatic carboxylic acids and lower aliphatic alcohols.

(b) A method of passing a gas containing at least one member selected from the group consisting of water, lower aliphatic carboxylic acids and lower aliphatic alcohols through the heteropolyacid salt supported catalyst obtained in the first step.

(c) A method of passing the heteropolyacid salt supported catalyst obtained in the first step, through an atmosphere of a gas containing at least one member selected from the group consisting of water, lower aliphatic carboxylic acids and lower aliphatic alcohols.

These methods may be used in combination of two or more thereof.

To speak more specifically on the method of performing the second step, for example, a method of filling the supported catalyst obtained in the first step into a vessel and contacting the gas therewith, or a method of filling the supported catalyst obtained in the first step into, in place of the vessel, a reactor where the production process of a lower aliphatic carboxylic acid ester is performed later, and contacting the gas therewith before feeding reaction starting materials, may be used.

With respect to the shape of the vessel or the reactor used here, any shape such as vertical type or horizontal type may be used without any particular limit.

In view of the time to be spent for re-filling the catalyst or the vessel cost, the preferred embodiment of the second step includes a method of filling the supported catalyst obtained in the first step into a reactor which is used in reacting a lower olefin with a lower aliphatic carboxylic acid in a gas phase to produce a lower aliphatic carboxylic acid ester, and then contacting therewith a gas containing at least one member selected from the group consisting of water, lower aliphatic carboxylic acids and lower aliphatic alcohols before feeding the reaction starting materials. At this time, the reaction may be performed in either a closed circulatory system or a flow system.

The second step is preferably performed under conditions not less than the dew point of the gas containing at least one member selected from the group consisting of water, lower aliphatic carboxylic acids and lower aliphatic alcohols. If the conditions are less than the dew point of the gas, a part of the gas may turn into a liquid. In this case, the heteropolyacid salt supported on the catalyst in the first step or other catalyst components supported, as desired, may dissolve out to change the catalyst composition, and in the worst case, the catalyst may be deactivated. As long as the catalyst is not adversely affected, the conditions in performing the second step are not particularly limited.

The preferred embodiment of the conditions at the dew point or more of the gas may vary depending on the composition of the gas or the pressure or the like in the practice of the step, however, the contact temperature is preferably from 80 to 300° C., more preferably from 100 to 260° C.

The contact pressure is not particularly limited and may be either normal pressure or raised pressure. The contact pressure is preferably from 0 to 3 MPaG (gauge pressure), more preferably from 0 to 2 MPaG (gauge pressure).

The lower aliphatic carboxylic acid in a gas containing at least one member selected from the group consisting of water, lower aliphatic carboxylic acids and lower aliphatic alcohols used in the second step, is preferably a lower aliphatic carboxylic acid having from 1 to 6 carbon atoms. Specific examples thereof include formic acid, acetic acid, propionic acid, n-butyric acid and isobutyric acid. Among these, preferred are acetic acid and propionic acid.

The lower aliphatic alcohol in a gas containing at least one member selected from the group consisting of water, lower aliphatic carboxylic acids and lower aliphatic alcohols used in the second step is preferably a lower aliphatic alcohol having from 1 to 6 carbon atoms. Specific examples thereof include methanol, ethanol, 1-propanol, 2-propanol, 1-butanol and 2-butanol. Among these, preferred are methanol, ethanol and n-propanol.

The composition of the gas containing at least one member selected from the group consisting of water, lower aliphatic carboxylic acids and lower aliphatic alcohols used in the second step is not particularly limited, and water, a lower aliphatic carboxylic acid and a lower aliphatic alcohol can be mixed at any ratio. The composition is preferably such that water:a lower aliphatic carboxylic acid:a lower aliphatic alcohol=1.0:0.1-10.0:0.1-5.0 by molar ratio.

The composition of the gas may be constant from the beginning to the end of contacting or may vary according to the contacting time or the stage of contacting.

The gas containing at least one member selected from the group consisting of water, lower aliphatic carboxylic acids and lower aliphatic alcohols used in the second step is more preferably water alone or a mixed gas of water and acetic acid, still more preferably a mixed gas of water and acetic acid because the effect can be provided within a short time.

In the case of using a mixed gas of water and acetic acid as the gas, the composition is not particularly limited but preferably such that water:acetic acid=1.0:0.1-10.0, more preferably water:acetic acid=1.0:0.5-5.0, by molar ratio.

The gas hourly space velocity (hereinafter referred to as “GHSV”), which is the speed of feeding the gas in performing the contact with a gas containing at least one member selected from the group consisting of water, lower aliphatic carboxylic acids and lower aliphatic alcohols used in the second step, is not particularly limited. The GHSV is preferably from 100 to 7,000 hr⁻¹, more preferably from 300 to 3,000 hr⁻¹.

If the GHSV is too high, the amount of the gas used may increase and this is not preferred in view of the cost. From this standpoint, the contacting may also be performed by feeding a constant amount of the gas and enclosing it in a vessel.

The contacting time is not particularly limited, however, it is preferably from 0.5 to 200 hours, more preferably from 0.5 to 100 hours, and most preferably from 0.5 to 50 hours. The optimal time varies depending on the composition and concentration of the gas, the temperature and pressure at the contacting, and the catalyst component.

Generally, if the contacting time is less than 0.5 hours, the effect of the second step may not be fully brought out, whereas if the contacting time is prolonged, the effect may be liable to increase but even if the contacting time is prolonged to exceed 200 hours, the effect may not increase any more, moreover, in the case where gas is contacted in the flowing state, the amount of the gas used may increase and this is not preferred in view of the profitability.

The present invention (II) is described below.

The present invention (II) is a process for producing a catalyst for use in producing a lower aliphatic carboxylic acid ester, the catalyst being used in reacting a lower olefin with a lower aliphatic carboxylic acid in a gas phase to produce a lower aliphatic carboxylic acid ester, which process comprises the following first and second steps:

First Step

a step for loading one or more heteropolyacid salts on a support to obtain a heteropolyacid salt supported catalyst; and

Second Step

a step for contacting the heteropolyacid salt supported catalyst obtained at the first step with a gas containing at least one member selected from the group consisting of water, lower aliphatic carboxylic acids and lower aliphatic alcohols to obtain a catalyst for use in producing a lower aliphatic carboxylic acid ester.

The heteropolyacid salt and the support used in the first step, the method for loading the heteropolyacid salt on a support performed in the first step, the method for measuring the amount of the heteropolyacid salt supported, and the preferred amount of the heteropolyacid salt supported on a support may be the same as in the present invention (I).

Furthermore, the method for contacting the catalyst with a gas containing at least one member selected from the group consisting of water, lower aliphatic carboxylic acids and lower aliphatic alcohols performed in the second step, the conditions therefor such as temperature, pressure, GHSV and time, the lower aliphatic carboxylic acid and lower aliphatic alcohol used for the gas, and the compositional ratio of the gas to which water is further added, may be the same as in the present invention (I).

In the process for producing a catalyst for use in producing a lower aliphatic carboxylic acid ester of the present invention (II), as long as the following first and second steps are contained, other steps may be provided before, after or during these steps, if desired.

First Step

A step for loading one or more heteropolyacid salts on a support to obtain a heteropolyacid salt supported catalyst.

Second Step

A step for contacting the heteropolyacid salt supported catalyst obtained in the first step with a gas containing at least one member selected from the group consisting of water, lower aliphatic carboxylic acids and lower aliphatic alcohols to obtain a catalyst for use in producing a lower aliphatic carboxylic acid ester.

Examples of the step which is provided, as desired, include a step for loading a third component so as to more improve the catalytic activity. In such a case, this loading operation may be performed simultaneously with the operation of loading the heteropolyacid salt in the first step, if possible.

After the second step of contacting with a gas containing at least one member selected from the group consisting of water, lower aliphatic carboxylic acids and lower aliphatic alcohols, the catalyst may further be contacted with another gas.

The present invention (III) is described below.

The present invention (III) is a process for producing a lower aliphatic carboxylic acid ester, comprising reacting a lower olefin with a lower aliphatic carboxylic acid in a gas phase in the presence of the catalyst for use in producing a lower aliphatic carboxylic acid ester of the present invention (I).

In the process for producing a lower aliphatic carboxylic acid ester of the present invention (III), a gas phase reaction is not particularly limited on the reaction form and the reaction may be performed in any form such as in a fixed bed system or fluidized bed system. The shape of the support which governs the shape or size of the catalyst may be selected in the range from powder to a compact formed into a size of several mm according to the reaction form employed.

Examples of the lower olefin which can be used in the process for producing a lower aliphatic carboxylic acid ester of the present invention (III), include ethylene, propylene, n-butene, isobutene and a mixture of two or more thereof.

The lower aliphatic carboxylic acid is suitably a carboxylic acid having from 1 to 4 carbon atoms and specific examples thereof include formic acid, acetic acid, propionic acid, butyric acid, acrylic acid and methacrylic acid.

The ratio between the lower olefin and the lower aliphatic carboxylic acid used as the starting materials is not particularly limited. In view of the conversion of the lower olefin, the lower olefin is preferably used in an equimolar or excess amount based on the lower aliphatic carboxylic acid. Specifically, the molar ratio of the lower olefin to the lower aliphatic carboxylic acid is preferably in the range of lower olefin:a lower aliphatic carboxylic acid=from 1:1 to 30:1, more preferably from 3:1 to 20:1, still more preferably from 5:1 to 15:1.

Furthermnore, from the standpoint of maintaining the catalytic activity, a small amount of water is preferably added to the starting materials. However, if an excessively large amount of water is added, the amount of a by-product such as an alcohol or an ether may disadvantageously increase. The amount of water added is preferably from 0.5 to 15 mol %, more preferably from 2 to 8 mol %, in terms of the molar ratio of water to the total amount of the lower olefin, the lower aliphatic carboxylic acid, as the starting materials, and water added.

The reaction conditions such as temperature and pressure preferred in the process for producing a lower aliphatic carboxylic acid ester of the present invention (III), may vary depending on the lower olefin and the lower aliphatic carboxylic acid used as the starting materials. The reaction conditions such as temperature and pressure are preferably combined so that the starting materials each can be kept in the gas state and the reaction can satisfactorily proceed.

In general, the temperature is preferably from 120 to 300° C., more preferably from 140 to 250° C. The pressure is preferably from 0 to 3 MPaG (gauge pressure), more preferably from 0 to 2 MPaG (gauge pressure).

The starting materials each is not particularly limited on the GHSV. If the GHSV is excessively high, the gas may pass through before the reaction satisfactorily proceeds, whereas if it is too low, problems, such as reduction in the productivity, may arise. The GHSV is preferably from 100 hr⁻¹ to 7,000 hr⁻¹, more preferably from 300 hr⁻¹ to 3,000 hr⁻¹.

The unreacted lower olefin and also alcohol and ether produced as by-products in the reaction may be recycled and used as they are. At this time, the substances which are harmful to the catalyst for use in producing a lower aliphatic carboxylic acid ester, such as butene and an aldehyde, are difficult to separate from olefin, alcohol, ether and the like, and may sometimes be transferred to a reactor. In such a case, the catalyst may be conspicuously reduced in activity or extremely shortened in the life. The process for producing a lower aliphatic carboxylic acid ester of the present invention (III) using the catalyst for use in producing a lower aliphatic carboxylic acid ester of the present invention (I) can greatly decrease the production of those by-products at the stage of reaction, therefore, the present invention (III) is particularly effective in the case where the above-described recycling system is employed in the production process.

The present invention (IV) is described below.

The present invention (IV) is a process for producing a lower aliphatic carboxylic acid ester, comprising reacting a lower olefin with a lower aliphatic carboxylic acid in a gas phase, which process comprises the following first to fourth steps:

First Step

a step of loading one or more heteropolyacid salts on a support to obtain a heteropolyacid salt supported catalyst;

Second Step

a step of filling the heteropolyacid salt supported catalyst obtained in the first step, into a reactor for use in the reaction of a lower olefin with a lower aliphatic carboxylic acid in a gas phase;

Third Step

a step of contacting the heteropolyacid salt supported catalyst filled in the reactor, with a gas containing at least one member selected from the group consisting of water, lower aliphatic carboxylic acids and lower aliphatic alcohols; and

Fourth Step

a step of passing a mixed gas containing the lower olefin and the lower aliphatic carboxylic acid through the heteropolyacid salt supported catalyst after the third step, to obtain the lower aliphatic carboxylic acid ester.

The first step is described below.

The first step in the process for producing a lower aliphatic carboxylic acid ester of the present invention (IV) is a step of loading a heteropolyacid salt on a support to obtain a heteropolyacid salt supported catalyst.

The heteropolyacid salt and the support used in the first step, the method for loading the heteropolyacid salt on a support performed in the first step, the method for measuring the amount of the heteropolyacid salt supported, and the preferred amount of the heteropolyacid salt supported on a support may be the same as in the present invention (I).

The second step is described below.

The second step in the process for producing a lower aliphatic carboxylic acid ester of the present invention (IV) is a step of filling the heteropolyacid salt supported catalyst obtained in the first step, into a reactor for use in reacting a lower olefin with a lower aliphatic carboxylic acid in a gas phase.

The second step is a step of filling the heteropolyacid salt supported catalyst into a reactor for use in the reaction of a lower olefin with a lower aliphatic carboxylic acid, so that the second step of the present invention (I) which is the catalyst for use in producing a lower aliphatic carboxylic acid ester, or the second step of the present invention (II) which is the process for producing a catalyst for use in producing a lower aliphatic carboxylic acid ester, namely, a step of contacting the heteropolyacid salt supported catalyst obtained in the first step with a gas containing at least one member selected from the group consisting of water, lower aliphatic carboxylic acids and lower aliphatic alcohols, can be performed in the reactor without using another vessel.

The reactor used in the second step is not particularly limited. A fixed bed gas phase-type reactor is preferred, and a reactor having a form of multi-tube system and/or multi-layer system is more preferred. In general, a reactor having a form of multi-tube system and/or multi-layer system is superior in the reaction results, thermal efficiency, eases of control and the like. Of course, the present invention is not limited thereto.

In the present invention, the term “filling the catalyst into a reactor” refers to placing the catalyst in a predetermined site of the reactor. The site and the method for the placement and in the case where the reactor uses a fixed bed system, the method for fixing the catalyst may vary depending on the form of the reactor and these are not particularly limited. Specific examples of the reactor include FIG. C.4.43 “Methanol Treating Gas Phase Reactor” in the item of “4) Fixed catalyst gas phase reactor”, described in Kagaku Sochi Binran (Chemical Apparatuses Handbook), 2nd ed., 3rd imp., pp. 905 to 906, edited by Society of Japan Chemical Engineering, published by Maruzen (Feb. 20, 1980).

The third step is described below.

The third step in the process for producing a lower aliphatic carboxylic acid ester of the present invention (IV) is a step of contacting a gas containing at least one member selected from the group consisting of water, lower aliphatic carboxylic acids and lower aliphatic alcohols with the heteropolyacid salt supported catalyst filled in the reactor.

The method for contacting the catalyst with a gas containing at least one member selected from the group consisting of water, lower aliphatic carboxylic acids and lower aliphatic alcohols performed in the third step, the conditions such as temperature, pressure, space velocity and time, the lower aliphatic carboxylic acid and lower aliphatic alcohol used for the gas, and the compositional ratio of the gas to which water is further added, may be the same as the second step of the present invention (I).

In performing the contacting in the reactor, the conditions therefor are not particularly limited, and the preferred conditions are considered to vary depending on the form, shape, size or constructive material of the reactor used for the contacting. In general, the contacting may be performed under the conditions described above for the second step of the present invention (I).

The fourth step is described below.

The fourth step of the present invention (IV) is a step of passing a mixed gas containing the lower olefin and the lower aliphatic carboxylic acid through the heteropolyacid salt supported catalyst—after the third step, to obtain the lower aliphatic carboxylic acid ester.

The lower olefin and lower aliphatic carboxylic acid used in the fourth step, the amount ratio thereof, the conditions such as addition of water, temperature, pressure and GHSV on performing the fourth step, and the recycling operation mainly of the unreacted lower olefin may be the same as in the process for the producing a lower aliphatic carboxylic acid ester of the present invention (III).

The third step and the fourth step of the present invention (IV) may or may not be clearly distinguished. Examples of the practical embodiment having a clear distinction between both steps include the case where after judging that the contacting in the third step is finished, the flow of the gas containing at least one member selected from the group consisting of water, lower aliphatic carboxylic acids and lower aliphatic alcohols is once stopped or the temperature is further lowered to complete the third step and, thereafter, the starting material gas containing a lower olefin and a lower aliphatic carboxylic acid is passed as the reaction gas in the fourth step.

Examples of the practical embodiment not having a clear distinction between the two steps include the case where, after judging that the contacting in the third step is finished, while not once stopping the flow of the gas containing at least one member selected from the group consisting of water, lower aliphatic carboxylic acids and lower aliphatic alcohols or by adjusting the temperature to the range suitable for the fourth step to prepare the initiation of reaction, the starting material gas containing a lower olefin and a lower aliphatic carboxylic acid is passed as the reaction gas in the fourth step.

For example, in the case where the lower aliphatic carboxylic acid used for the contacting in the third step is a lower aliphatic carboxylic acid corresponding to the ester as an objective product of the process for producing a lower aliphatic carboxylic acid ester of the present invention (IV), the reaction may be performed according to the practical embodiment not having a clear distinction between the third step and the fourth step. On the other hand, in the case where the lower aliphatic carboxylic acid used for the contacting in the third step is not a lower aliphatic carboxylic acid corresponding to the objective ester, the reaction is preferably performed by providing a clear distinction between those two steps.

In general, the contacting in the third step is preferably performed using a lower aliphatic carboxylic acid corresponding to the ester as an objective product of the process for producing a lower aliphatic carboxylic acid ester of the present invention (IV). For example, in the case of producing ethyl acetate as the lower aliphatic carboxylic acid ester by applying the process for producing a lower fatty acid ester of the present invention (IV), one of the preferred practical embodiments is a method of performing the contacting in the third step using a mixed gas of water and acetic acid for a constant time under constant conditions, then varying various conditions within the reactor, such as temperature, pressure and GHSV, to fit the production process of a lower aliphatic carboxylic acid ester, and adding ethylene to the gas introduced into the reactor. Of course, the present invention (IV) is not limited thereto.

### B. The Inventions (V) to (XI)

The present inventors have made extensive investigations for a catalyst supported on a siliceous support and used in the production of a lower aliphatic carboxylic acid ester from a lower olefin and a lower aliphatic carboxylic acid, which can prevent a great reduction in the catalytic activity, is protected from cracking or abrasion during the production of a lower aliphatic carboxylic acid ester and can ensure stable operation. As a result, it has been found that the crush strength of a catalyst is closely related to the silicon purity of a siliceous support and when the content of silicon in the siliceous support falls within a predetermined range, the obtained catalyst for producing a lower aliphatic carboxylic acid ester can prevent a great reduction in the catalytic activity, is protected from cracking or abrasion during the production of a lower aliphatic carboxylic acid ester and can be stably operated. The present invention has been accomplished based on this finding.

In the siliceous support of the present invention, the silicon content is from 39.7 to 46.3% by mass (from 85 to 99% by mass in terms of silicon dioxide), preferably from 41.1 to 46.3% by mass (from 88 to 99% by mass in terms of silicon dioxide), more preferably from 42.1 to 46.3% by mass (from 90 to 99% by mass in terms of silicon dioxide). According to the present invention, the support having a silicon content within the above-described range is found to have a crush strength of 30 N or more, whereby the objects of the present invention can be attained.

The silicon content of the support can be measured by a chemical analysis such as inductively coupled plasma emission spectrometry (ICP), fluorescent x-ray spectrometry and atomic absorption spectrometry. In these methods, the silicon content is generally measured as a silicon dioxide content. In the present invention, a silicon dioxide content measured by ICP out of these methods is preferably used, however, a value according to the value measured by other methods or a value obtained by extrapolating the measured value may also be used.

The process for producing the support of the present invention is not particularly limited and may be any process. Specific examples thereof are described in Zoryu Handbook (Granulation Handbook), edited by Nippon Funtai Kogyo Gijutsu Kyokai, published by Ohm Kabushiki Kaisha on Mar. 10, 1991, pp.661-671, but are not limited thereto.

The support of the present invention is not limited on the shape thereof and may have any shape. For example, a support in a powder, a spherical, a pellet-like or any other arbitrary form may be used. A support having a spherical or a pellet-like form is preferred. Also, the particle size is not particularly limited. Although the preferred particle size varies depending on the reaction method, in the case of use in a fixed bed system, the particle size is preferably from 2 to 10 mm, more preferably from 3 to 7 mm, and in the case of use in a fluidized bed system, the preferred range is from a powder to a particle size of 5 mm, more preferably from powder to a particle size of 2 mm.

According to the present invention, a catalyst for producing a lower aliphatic carboxylic acid ester is also provided, which is supported on the above-described support of the present invention and is used for producing a lower aliphatic carboxylic acid ester by reacting a lower olefin with a lower aliphatic carboxylic acid in a gas phase. In this catalyst, a crush strength equal to the crush strength of the support can be maintained and therefore, the catalyst for producing a lower aliphatic carboxylic acid ester of the present invention also has a crush strength of 30 N or more.

The catalyst for producing a lower aliphatic carboxylic acid ester of the present invention can be produced, for example, by a process comprising a step of loading at least one member selected from heteropolyacids and salts thereof on the above-described support of the present invention.

The heteropolyacids which can be used in the production of the catalyst of the present invention comprise a center element and a peripheral element to which oxygen is bonded. The center element is usually silicon or phosphorus but may comprise an arbitrary element selected from various kinds of elements belonging to Groups 1 to 17 of the Periodic Table. Specific examples thereof include cupric ion; divalent beryllium, zinc, cobalt and nickel ions; trivalent boron, aluminum, gallium, iron, cerium, arsenic, antimony, phosphorus, bismuth, chromium and rhodium ions; tetravalent silicon, germanium, tin, titanium, zirconium, vanadium, sulfur, tellurium, manganese, nickel, platinum, thorium, hafnium, cerium ions and other rare earth ions; pentavalent phosphorus, arsenic, vanadium and antimony ions; hexavalent tellurium ion; and heptavalent iodide ion, however, the present invention is by no means limited thereto. Specific examples of the peripheral element include tungsten, molybdenum, vanadium, niobium and tantalum, however, the present invention is by no means limited thereto.

These heteropolyacids are also known as a “polyoxo-anion”, a “polyoxometallic salt” or a “metal oxide cluster”. The structures of some well-known anions are named after the researcher in this field and called, for example, Keggin, Wells-Dawson or Anderson-Evans-Perloff structures. These are described in detail in “Poly-San no Kagaku, Kikan Kagaku Sosetsu (Chemistry of Polyacids, Quarterly of Chemistry General View)”, No. 20, edited by Nippon Kagaku Kai (1993). The heteropolyacids usually have a high molecular weight, for example, a molecular weight of 700 to 8,500, and include not only the monomers but also dimeric complexes thereof.

Specific examples of the heteropolyacids include:

| | |
|---|---|
| Tungstosilicic acid | H₄[SiW₁₂O₄₀].xH₂O |
| Tungstophosphoric acid | H₃[PW₁₂O₄₀].xH₂O |
| Molybdophosphoric acid | H₃[PMO₁₂O₄₀]xH₂O |
| Molybdosilicic acid | H₄[SiMo₁₂O₄₀].xH₂O |
| Vanadotungstosilicic acid | H₄₊ₙ[SiVₙW₁₂₋ₙO₄₀].xH₂O |
| Vanadotungstophosphoric acid | H₃₊ₙ[PVₙW₁₂₋ₙO₄₀].xH₂O |
| Vanadomolybdophosphoric acid | H₃₊ₙ[PVₙMo₁₂₋ₙO₄₀].xH₂O |
| Vanadomolybdosilicic acid | H₄₊ₙ[SiVₙMO₁₂₋ₙO₄₀].xH₂O |
| Molybdotungstosilicic acid | H₄[SiMOₙW₁₂₋ₙO₄₀].xH₂O |
| Molybdotungstophosphoric acid | H₃[PMOₙW₁₂₋ₙO₄₀].xH₂O |

wherein n is an integer of 1 to 11 and x is an integer of 1 or more. However, the present invention is by no means limited thereto.

Among these, preferred are tungstosilicic acid, tungstophosphoric acid, molybdophosphoric acid, molybdosilicic acid, vanadotungstosilicic acid and vanadotungstophosphoric acid, more preferred are tungstosilicic acid, tungstophosphoric acid, vanadotungstosilicic acid and vanadotungstophosphoric acid.

The synthesis method for these heteropolyacids is not particularly limited and any method may be used. For example, the heteropolyacid may be obtained by heating an acidic aqueous solution containing a salt of molybdic acid or tungstic acid and a simple oxygen acid of hetero atom or a salt thereof (pH: about 1 to 2). For isolating the heteropolyacid compound from the aqueous heteropolyacid solution produced, a method of crystallizing and separating the compound in the form of a metal salt may be used. Specific examples thereof include those described in Shin Jikken Kagaku Koza 8, Muki Kagoubutsu no Gosei (III) (New Experimental Chemistry Course 8, Synthesis of Inorganic Compounds (III)), 3rd ed., page 1413, edited by Nippon Kagaku Kai, published by Maruzen on Aug. 20, 1984, however, the present invention is not limited thereto. The Keggin structure of the heteropolyacid synthesized may be identified by the chemical analysis or by the X-ray diffraction or UV or IR measurement.

The heteropolyacids, particularly in the case where the heteropolyacids are free acids or are some salts, have a relatively high solubility in polar solvents such as water and other oxygen-containing solvents, and the solubility can be controlled by appropriately selecting the counter ion.

The heteropolyacids can be loaded on a support by allowing a solution or suspension obtained by dissolving or suspending a heteropolyacid in a solvent, to be absorbed into the support.

The amount of a heteropolyacid supported is preferably from 10 to 150% by mass, more preferably from 30 to 100% by mass, based on the total weight of the support. If the heteropolyacid content is less than 10% by mass, the active component content of the catalyst is too small and the activity per unit weight of catalyst may disadvantageously decrease. If the heteropolyacid content exceeds 150% by mass, the effective pore volume decreases and, as a result, the effect of the increase in the supported amount may not be brought out and at the same time, coking is disadvantageously liable to occur to seriously shorten the catalyst life.

The heteropolyacid salts which can be used in the production of the catalyst of the present invention may be a metal salt or an onium salt resulting from substituting a part or all of the hydrogen atoms of a heteropolyacid. Specific examples thereof include metal salts such as lithium, sodium, magnesium, barium, copper, gold and gallium, and onium salts, however, the present invention is not limited thereto. Among these, lithium salts, sodium salts, gallium salts, copper salts and gold salts are preferred, and lithium salts, sodium salts and copper salts are more preferred.

Examples of the starting material for the element of forming a heteropolyacid salt include lithium nitrate, lithium acetate, lithium sulfate, lithium sulfite, lithium carbonate, lithium phosphate, lithium oxalate, lithium nitrite, lithium chloride, lithium citrate, sodium nitrate, sodium acetate, sodium sulfate, sodium carbonate, monosodium phosphate, disodium phosphate, sodium oxalate, sodium nitrite, sodium chloride, sodium citrate, magnesium nitrate hexahydrate, magnesium acetate tetrahydrate, magnesium sulfate, magnesium carbonate, magnesium phosphate tricosahydrate, magnesium oxalate dihydrate, magnesium chloride, magnesium citrate, barium nitrate, barium acetate, barium sulfate, barium carbonate, barium hydrogenphosphate, barium oxalate monohydrate, barium sulfite, barium chloride, barium citrate, copper nitrate, copper acetate, copper sulfate, copper carbonate, copper diphosphate, copper oxalate, copper chloride, copper citrate, aurous chloride, chloroauric acid, auric oxide, auric hydroxide, auric sulfide, aurous sulfide, gallium dichloride, gallium monochloride, gallium citrate, gallium acetate, gallium nitrate, gallium sulfate, gallium phosphate, ammonium acetate, ammonium carbonate, ammonium nitrate, ammonium dihydrogenphosphate, ammonium hydrogencarbonate, ammonium citrate, ammonium nitrate, diammonium phosphate, monoammonium phosphate and ammonium sulfate, however, the present invention is by no means limited thereto.

Among these, preferred are lithium nitrate, lithium acetate, lithium carbonate, lithium oxalate, lithium citrate, sodium nitrate, sodium acetate, sodium carbonate, sodium oxalate, sodium citrate, copper nitrate, copper acetate, copper carbonate, copper citrate, aurous chloride, chloroauric acid, gallium citrate, gallium acetate and gallium nitrate, and more preferred are lithium nitrate, lithium-acetate, lithium carbonate, lithium oxalate, lithium citrate, sodium nitrate, sodium acetate, sodium carbonate, sodium oxalate, sodium citrate, copper nitrate, copper acetate, copper carbonate and copper citrate.

Specific examples of the heteropolyacid salts include lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of molybdophosphoric acid, sodium salt of molybdophosphoric acid, copper salt of molybdophosphoric acid, gold salt of molybdophosphoric acid, gallium salt of molybdophosphoric acid, lithium salt of molybdosilicic acid, sodium salt of molybdosilicic acid, copper salt of molybdosilicic acid, gold salt of molybdosilicic acid, gallium salt of molybdosilicic acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid, gallium salt of vanadotungstophosphoric acid, lithium salt of vanadomolybdophosphoric acid, sodium salt of vanadomolybdophosphoric acid, copper salt of vanadomolybdophosphoric acid, gold salt of vanadomolybdophosphoric acid, gallium salt of vanadomolybdophosphoric acid, lithium salt of vanadomolybdosilicic acid, sodium salt of vanadomolybdosilicic acid, copper salt of vanadomolybdosilicic acid, gold salt of vanadomolybdosilicic acid and gallium salt of vanadomolybdosilicic acid.

Among these, preferred are lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of molybdophosphoric acid, sodium salt of molybdophosphoric acid, copper salt of molybdophosphoric acid, gold salt of molybdophosphoric acid, gallium salt of molybdophosphoric acid, lithium salt of molybdosilicic acid, sodium salt of molybdosilicic acid, copper salt of molybdosilicic acid, gold salt of molybdosilicic acid, gallium salt of molybdosilicic acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid and gallium salt of vanadotungstophosphoric acid.

More preferred are lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid and gallium salt of vanadotungstophosphoric acid.

The method for loading a heteropolyacid salt on a support roughly includes the following three methods (1) to (3):

(1) a method of loading a desired heteropolyacid on a support and then loading a starting material for the desired element for forming a salt,

(2) a method of loading a desired heteropolyacid and a starting material for the element of forming a salt, together on a support or loading a previously prepared heteropolyacid salt, and

(3) a method of previously loading a starting material for the element for forming a salt on a support and then loading a desired heteropolyacid.

In any of these methods (1) to (3), the heteropolyacid, a salt thereof and the starting material for the element of forming a salt each can be loaded on a support after dissolving it or suspending it in an appropriate solvent. The solvent may be any solvent as long as it can uniformly dissolve or suspend the desired heteropolyacid, a salt thereof and the starting material for the element of forming a salt, and examples of the solvent which can be used include water, an organic solvent and a mixture thereof. Among these, preferred are water, alcohol and carboxylic acid.

The method for dissolving or suspending the desired heteropolyacid, a salt thereof and the starting material for the element of forming a salt may also be any method as long as it can uniformly dissolve or suspend the materials. In the case of a free acid, a free acid which can dissolve may be dissolved as it is in a solvent and even in the case of a free acid which cannot completely dissolve, if the free acid can be uniformly suspended by forming it into fine powder, the free acid may be suspended as such.

In the method (1), a solution or suspension obtained by dissolving or suspending a heteropolyacid in a solvent is absorbed into a support to thereby load the heteropolyacid on the support and then, a solution or suspension of a starting material for the element of forming a desired salt is absorbed into the support to thereby load the element. At this time, a neutralization reaction proceeds on the support and, as a result, a catalyst having supported thereon a heteropolyacid salt can be prepared.

In the method (2), a heteropolyacid and a starting material for the element of forming a salt are dissolved or suspended together or these are dissolved or suspended separately and then mixed, and the thus-prepared solution or suspension is absorbed into a support and thereby loaded on the support. If the compound is in the state of a heteropolyacid salt, a uniform solution or suspension may be obtained in the same manner as in the case of a free acid.

In the method (3), a solution or suspension of a starting material for the element of forming a salt is previously prepared, the solution or suspension is absorbed into a support to thereby load the element, and then a desired heteropolyacid is loaded thereon. This method includes a method of using an element which is previously contained in the support and can form a heteropolyacid salt.

More specifically, a part or all of the elements previously contained in a support sometimes act to form a salt of a heteropolyacid on loading and, as a result, a heteropolyacid salt is formed. Examples of such an element include potassium, sodium, calcium, iron, magnesium, titanium and aluminum, however, the present invention is not limited thereto.

The kind of the element previously contained in a support and the amount thereof can be measured by chemical analysis such as ICP, fluorescent x-ray spectrometry and atomic absorption spectrometry. The kind and the amount of the element vary depending on the support, however, potassium, sodium, calcium, iron, magnesium, titanium and ammonium are sometimes contained in a relatively large amount and the content thereof is approximately from 0.001 to 5.0% by mass. Therefore, depending on the combination of a support and a heteropolyacid, the element may be previously contained in the support in an amount large enough to form a salt, though this may vary depending on the kind and the amount of the heteropolyacid supported.

The amount of a heteropolyacid salt supported is preferably from 10 to 150% by mass, more preferably from 30 to 100% by mass, based on the total weight of the support. If the heteropolyacid salt content is less than 10% by mass, the active component content of the catalyst is too small and the activity per unit weight of catalyst may disadvantageously decrease. If the heteropolyacid salt content exceeds 150% by mass, the effective pore volume decreases and, as a result, the effect of the increase in the supported amount may not be brought out and, at the same time, coking is disadvantageously liable to occur to seriously shorten the catalyst life.

The method for loading a solution or suspension of a heteropolyacid and/or a heteropolyacid salt on a support is not particularly limited and a known method may be used. More specifically, for example, the catalyst may be prepared by dissolving a heteropolyacid in distilled water corresponding to the liquid absorption amount of a support used and impregnating the solution into the support. Also, the catalyst may be prepared by using an excess aqueous solution, dipping a support in the heteropolyacid solution while appropriately moving the support and then removing the excess acid through filtration. The volume of the solution or suspension used at this time varies depending on the support or loading method used.

The thus-obtained wet catalyst is suitably dried by placing it in a heating oven for a few hours. The drying method is not particularly limited and any method such as standing or belt conveyor may be used. After the drying, the catalyst is cooled to the ambient temperature in a desiccator so as not to absorb moisture.

The amount of a heteropolyacid and/or a heteropolyacid salt supported in the thus-obtained heteropolyacid salt supported catalyst can be simply calculated by subtracting the weight of the support used from the weight after drying of the catalyst prepared. To be more exact, the supported amount can be determined by chemical analysis such as ICP, fluorescent X-ray spectrometry and atomic absorption spectrometry.

The catalyst for producing a lower aliphatic carboxylic acid ester of the present invention is preferably produced by a production process comprising a first step of loading at least one member selected from the group consisting of heteropolyacids and salts thereof on a support of the present invention to obtain a catalyst, and a second step of contacting this catalyst with a gas containing at least one member selected from the group consisting of water, lower aliphatic carboxylic acids and lower aliphatic alcohols to obtain a catalyst for the production of a lower aliphatic carboxylic acid ester.

In this method, the loading of a heteropolyacid and/or a heteropolyacid salt on a support in the first step can be performed according to the method described in detail above.

The second step in the above-described production process is a step of contacting the catalyst having supported thereon a heteropolyacid and/or a heteropolyacid salt, which is obtained in the first step, with a gas containing at least one member selected from the group consisting of water, lower aliphatic carboxylic acids and lower aliphatic alcohols.

The method for contacting the supported catalyst obtained in the first step with a gas containing at least one member selected from the group consisting of water, lower aliphatic carboxylic acids and lower aliphatic alcohols is not particularly limited and, for example, the following methods may be used:

(a) a method of placing the catalyst, obtained in the first step, in an atmosphere of gas containing at least one member selected from the group consisting of water, lower aliphatic carboxylic acids and lower aliphatic alcohols;

(b) a method of passing the catalyst obtained in the first step, through a gas containing at least one member selected from the group consisting of water, lower aliphatic carboxylic acids and lower aliphatic alcohols; and

(c) a method of passing the catalyst obtained in the first step, through an atmosphere of gas containing at least one member selected from the group consisting of water, lower aliphatic carboxylic acids and lower aliphatic alcohols. These methods may be used in a combination of two or more thereof, if desired.

To speak more specifically regarding the method of performing the second step, for example, a method of filling the catalyst obtained in the first step into a vessel and contacting the above-described gas therewith, or a method of filling the catalyst obtained in the first step into, in place of the vessel, a reactor where the production process of a lower aliphatic carboxylic acid ester is performed later, and contacting the above-described gas therewith before feeding reaction starting materials, may be used. With respect to the shape of the vessel or the reactor used here, any shape such as vertical type or horizontal type may be used without any particular limit.

In view of the time period necessary for re-filling the catalyst or the vessel cost, the preferred embodiment of the second step includes a method of filling the catalyst obtained in the first step into a reactor which is used at the time of reacting a lower olefin with a lower aliphatic carboxylic acid in a gas phase to produce a lower aliphatic carboxylic acid ester, and then contacting therewith a gas containing at least one member selected from the group consisting of water, lower aliphatic carboxylic acids and lower aliphatic alcohols, generally in the absence of any lower olefin, and thus before feeding the reaction starting materials. This method may be performed in either a closed circulatory system or a flow system.

The second step is preferably performed under a condition higher than the dew point of the gas containing at least one member selected from the group consisting of water, lower aliphatic carboxylic acids and lower aliphatic alcohols. If the condition is less than the dew point of this gas, a part of the gas may turn into a liquid. In this case, a heteropolyacid and/or a heteropolyacid salt supported on the catalyst in the first step, or other catalyst components supported if desired, may dissolve out to change the catalyst composition and in the worst case, the catalyst may be deactivated. Insofar as the catalyst is not adversely affected, the conditions in performing the second step are not particularly limited.

The preferred embodiment of a condition higher than the dew point of the above-described gas may vary depending on the composition of the gas or the pressure or the like in the practice, however, the contact temperature is preferably from 80 to 300° C., more preferably from 100 to 260° C.

The contact pressure is not particularly limited and may be either normal pressure or an applied pressure. The contact pressure is preferably from 0 to 3 MPaG (gauge pressure), more preferably from 0 to 2 MPaG (gauge pressure).

The lower aliphatic carboxylic acid in the gas containing at least one member selected from the group consisting of water, lower aliphatic carboxylic acids and lower aliphatic alcohols used in the second step is preferably a lower aliphatic carboxylic acid having from 1 to 6 carbon atoms. Specific examples thereof include formic acid, acetic acid, propionic acid, n-butyric acid and isobutyric acid. Among these, preferred are acetic acid and propionic acid.

The lower aliphatic alcohol in the gas containing at least one member selected from the group consisting of water, lower aliphatic carboxylic acids and lower aliphatic alcohols used in the second step is preferably a lower aliphatic alcohol having from 1 to 6 carbon atoms. Specific examples thereof include methanol, ethanol, 1-propanol, 2-propanol, 1-butanol and 2-butanol. Among these, particularly preferred are methanol, ethanol and n-propanol.

The composition of the gas containing at least one member selected from the group consisting of water, lower aliphatic carboxylic acids and lower aliphatic alcohols used in the second step is not particularly limited, and water, a lower aliphatic carboxylic acid and/or a lower aliphatic alcohol can be mixed at an arbitrary ratio. The composition is preferably such that water:lower aliphatic carboxylic acid:lower aliphatic alcohol=1.0:0.1 to 10.0:0.1 to 5.0 in terms of the molar ratio. The composition of the gas may be constant from the beginning to the end of contacting or may be changed according to the contact time or the stage of contacting.

The gas containing at least one member selected from the group consisting of water, lower aliphatic carboxylic acids and lower aliphatic alcohols used in the second step is more preferably water alone or a mixed gas of water and acetic acid, still more preferably a mixed gas of water and acetic acid, because a predetermined effect can be obtained within a short period of time. In the case of using a mixed gas of water and acetic acid, the composition thereof is not particularly limited but preferably such that water:acetic acid=1.0:0.1 to 10.0, more preferably water:acetic acid=1.0:0.5 to 5.0, in terms of the molar ratio.

The gas hourly space velocity (GHSV) of the gas, which is the speed of feeding the gas in performing the contact with a gas containing at least one member selected from the group consisting of water, lower aliphatic carboxylic acids and lower aliphatic alcohols in the second step, is not particularly limited. The GHSV is preferably from 100 to 7,000 hr⁻¹, more preferably from 300 to 3,000 hr⁻¹. If the GHSV is too high, the amount of the gas used increases and this is not preferred in view of the cost. From this standpoint, the contacting may also be performed in the state such that the gas is fed in a constant amount and enclosed in a vessel.

The contact time is not particularly limited but preferably from 0.5 to 200 hours, more preferably from 0.5 to 100 hours, and most preferably from 0.5 to 50 hours. The optimal contact time varies depending on the composition and concentration of the gas, the temperature and pressure at the contacting, and the catalyst components.

Generally, if the contact time is less than 0.5 hours, the effect of the second step may not be fully brought out, whereas if the contact time is prolonged, the effect is liable to increase, however, even if the contact time is prolonged to exceed 200 hours, the effect does not increase any more and, moreover, in the case where gas is contacted in the flowing state, the amount of the gas used increases and this is not preferred in view of the profitability.

These first and second steps may be performed either continuously or completely independently of each other. More specifically, for example, after purchasing the catalyst passed through the first step, the second step may be performed using this catalyst.

Furthermore, in the process for producing the catalyst for producing a lower aliphatic carboxylic acid ester according to the present invention, other steps may be provided, if desired. Such a step may be performed before, after or during the loading of a heteropolyacid and/or a heteropolyacid salt on a support, before the first step, between the first step and the second step, after the second step, or at any stage during these steps.

Examples of other steps performed if desired include a step of loading a third component having a purpose of more improving the catalyst performance. In this case, if possible, this loading operation and the operation of loading a heteropolyacid and/or a heteropolyacid salt be performed simultaneously. Furthermore, further contact with another gas may also be performed after the contact with the gas containing at least one member selected from the group consisting of water, lower aliphatic carboxylic acids and lower aliphatic alcohols.

The present invention also provides a process for producing a lower aliphatic carboxylic acid ester, comprising reacting a lower olefin with a lower aliphatic carboxylic acid in a gas phase in the presence of the catalyst for producing a lower aliphatic carboxylic acid ester of the present invention.

In practicing this process, the reaction form of the gas phase reaction is not particularly limited and any form such as fixed bed system and fluidized bed system may be employed. With respect to the shape of the support governing the shape and size of the catalyst, as described above, any desired shape can be selected according to the reaction form practiced.

Examples of the lower olefin which can be used in the process for producing a lower aliphatic carboxylic acid ester of the present invention include ethylene, propylene, n-butene, isobutene and a mixture of two or more thereof.

The lower aliphatic carboxylic acid is suitably a carboxylic acid having from 1 to 4 carbon atoms and specific examples thereof include formic acid, acetic acid, propionic acid, butyric acid, acrylic acid and methacrylic acid.

The proportion between the lower olefin and the lower aliphatic carboxylic acid used as the starting materials is not particularly limited. In view of the conversion of the lower olefin, the lower olefin is preferably used in an equimolar or excess molar amount to the lower aliphatic carboxylic acid. To speak specifically, the molar ratio of the lower olefin to the lower aliphatic carboxylic acid is preferably in the range of lower olefin:lower aliphatic carboxylic acid=from 1:1 to 30:1, more preferably from 3:1 to 20:1, still more preferably from 5:1 to 15:1.

In the process for producing a lower aliphatic carboxylic acid ester of the present invention, a slight amount of water is preferably added to the starting materials comprising a lower olefin and a lower aliphatic carboxylic acid from the standpoint of maintaining the catalytic activity. However, if an excessively large amount of water is added, the amount of by-products such as alcohol and ether disadvantageously increases. The amount of water added is preferably, in terms of the molar ratio of water to the sum total of lower olefin and lower aliphatic monocarboxylic acid as starting materials and water added, from 0.5 to 15 mol %, more preferably from 2 to 8 mol %.

In this process, preferred reaction conditions such as temperature and pressure vary depending on the kinds of the lower olefin and lower aliphatic carboxylic acid used as the starting materials. The reaction conditions such as temperature and pressure are preferably combined so that the starting materials can each be kept in the gas state and the reaction can satisfactorily proceed. In general, the temperature is preferably from 120 to 300° C., more preferably from 140 to 250° C. The pressure is preferably from 0 to 3 MPaG (gauge pressure), more preferably from 0 to 2 MPaG (gauge pressure).

The each starting material is not particularly limited regarding the GHSV, however, if the GHSV is excessively high, the starting materials pass through before the reaction satisfactorily proceeds, whereas if it is too low, there may arise problems such as reduction in the productivity. The GHSV is preferably from 100 to 7,000 hr⁻¹, more preferably from 300 to 3,000 hr⁻¹.

The unreacted lower olefin, and also the alcohol and the ether as by-products in the reaction may be recycled and used as they are. At this time, substances harmful to the catalyst for the production of a lower aliphatic carboxylic acid ester, such as butene and aldehyde, are difficult to separate from olefin, alcohol, ether and the like and may be sent to the reactor. If this is so, the catalyst performance may be seriously reduced or the life thereof may be extremely shortened. Accordingly, a catalyst, for producing a lower aliphatic carboxylic acid ester of the present invention, and which can greatly reduce the production of these by-products at the reaction stage, is used. The process for producing a lower aliphatic carboxylic acid ester of the present invention is effective particularly when the above-described recycling system is included in the production process.

The present invention will be further illustrated below by referring to the Examples and Comparative Examples, however, the present invention should not be construed as being limited thereto.

In the following Examples 1 to 16 and Comparative Examples 1 to 9, the measured values were obtained by analysis or measurements performed according to the following methods.

Conditions for Analysis of Metal in Support

The analysis of metals in the support was performed using a fluorescent x-ray analyzer (PW2404, manufactured by PHILIPS). Measurement conditions—atmosphere: helium, effective diameter: 25.0 mm, and matrix: 2SiO₂.

Conditions for Analysis of Uncondensed Gas

The analysis was performed under the following conditions using an absolute calibration curve process by sampling 50 ml of the effluent gas and passing the entire amount thereof into a 1 ml gas sampler attached to the gas chromatograph.

1. Ether, Carboxylic Acid Ester, Alcohol and Trace by-Products

Gas chromatography:

gas chromatography (GC-14B, manufactured by Shimadzu Seisakusho Co.) with a gas sampler for Shimadzu gas chromatograph (MGS-4, measuring tube: 1 ml)

Column: packed column SPAN 80, 15% Shinchrom A of 60 to 80 mesh (length: 5 m)

Carrier gas: nitrogen (flow rate: 25 ml/min)

Temperature conditions:

The detector and the vaporization chamber were at a temperature of 120° C., and the column temperature was 65° C. and constant.

Detector: FID (H₂ pressure: 60 kPa, air pressure: 100 kPa)

2. Butene

Gas chromatography:

gas chromatography (GC-14B, manufactured by Shimadzu Seisakusho Co.) with a gas sampler for Shimadzu gas chromatograph (MGS-4, measuring tube: 1 ml)

Column: packed column Unicarbon A-400 of 80/100 mesh, length: 2 m

Carrier gas: helium (flow rate: 23 ml/min)

Temperature condition:

The detector and the vaporization chamber were at a temperature of 130° C., and the column temperature was elevated from 40° C. to 95° C. at a temperature rising rate of 40° C./min.

Detector: FID (H₂ pressure: 70 kPa, air pressure: 100 kPa)

3. Ethylene

Gas chromatography:

gas chromatography (GC-14B, manufactured by Shimadzu Seisakusho Co.) with a gas sampler for Shimadzu gas chromatograph (MGS-4, measuring tube: 1 ml)

Column: packed column Unibeads IS, length: 3 m

Carrier gas: helium (flow rate: 20 ml/min)

Temperature condition:

The detector and the vaporization chamber were at a temperature of 120° C., and the column temperature was 65° C. and constant.

Detector: TCD (He pressure: 70 kPa, current: 90 mA, temperature: 120° C.)

Analysis of Solution Collected.

The analysis was performed using an internal standard method by injecting 0.4 μl of an analysis solution obtained by adding 1 ml of 1,4-dioxane as an internal standard to 10 ml of the reaction solution.

Gas chromatography:

GC-14B manufactured by Shimadzu Seisakusho Co.

Column: capillary column TC-WAX (length: 30 m, inner diameter: 0.25 mm, film thickness: 0.25 μm)

Carrier gas: nitrogen (split ratio: 20, column flow rate: 1 ml/min)

Temperature condition:

The detector and the vaporization chamber were at a temperature of 200° C., and the column temperature was kept at 50° C. for 5 minutes from the starting of analysis, then elevated to 150° C. at a temperature rising rate of 20° C./min, and kept at 150° C. for 10 minutes.

Detector: FID (H₂ pressure: 70 kPa, air pressure: 100 kPa).

Examples 1 to 16 and Comparative Examples 1 to 9

Support

Support 1: synthetic silica (N-602T, produced by Nikki Kagaku K.K.) (specific surface area: 132 m²/g, pore volume: 0.7 cm³/g)

Support 2: synthetic silica (CARiACT Q-10, produced by Fuji Silicia Kagaku K.K.) (specific surface area: 219.8 m²/g, pore volume: 0.660 cm³/g)

Support 3: natural silica (KA-160, produced by Sud Chemie AG) (specific surface area: 130 m²/g, pore volume: 0.53 cm³/g)

Support 4: natural silica (KA-0, produced by Sud Chemie AG) (specific surface area: 68.5 m²/g, pore volume: 0.71 cm3/g)

Support 5: silica gel (Wakogel C-200, produced by Wako Junyaku Kogyo K.K.) (specific surface area: 762 m²/g, pore volume: 0.23 cm³/g)

The metal analysis results of each support are shown in Table 1.

TABLE 1

| | | SiO₂ | K₂O | Na₂O | CaO | Cr₂O₃ | Fe₂O₃ | MgO | ZrO₂ | TiO₂ | Al₂O₃ | SrO | Nb₂O₅ | Rb₂O |
|---|---|---|---|---|---|---|---|---|---|---|---|---|---|---|
| 1 | Support 1 | 99.316 | 0.009 | 0.319 | 0.112 | 0.002 | 0.032 | 0.052 | 0.010 | 0.028 | 0.120 | 0.001 | — | — |
| 2 | Support 2 | 99.863 | — | 0.021 | 0.040 | — | 0.005 | 0.040 | 0.005 | 0.025 | — | — | — | — |
| 3 | Support 3 | 97.142 | 0.276 | 0.008 | 0.109 | 0.002 | 0.284 | 0.178 | 0.040 | 0.234 | 1.720 | — | 0.004 | 0.003 |
| 4 | Support 4 | 98.963 | 0.057 | 0.049 | 0.085 | 0.001 | 0.113 | 0.077 | 0.023 | 0.180 | 0.445 | — | 0.005 | 0.001 |
| 5 | Support 5 | 99.917 | — | — | 0.032 | — | — | 0.020 | 0.005 | 0.027 | — | — | — | — |

Unit: mass %

Preparation Process of Catalyst 1

Support 1 was preliminarily dried for 4 hours in a (hot-air type) drier previously adjusted to 110° C. After the preliminary drying, 1 liter of the support was measured on the bulk density using a 1 liter measuring cylinder. Tungstosilicic acid was weighed to the amount shown in Table 2 and after adding thereto 15 ml of pure water, uniformly dissolved. To this impregnating solution, pure water was further added to make the liquid amount shown in Volume of Solution Prepared of Table 2. Thereafter, the preliminarily dried support was weighed to the amount shown in Table 2, added to the impregnating solution and impregnated with the solution while thoroughly stirring. The support impregnated with the solution was transferred to a porcelain dish, air-dried for 1 hour and then dried in a hot-air type drier adjusted to 150° C. for 5 hours. After the drying, the catalyst was transferred in a desiccator and left standing to cool to room temperature. This is designated as a “tungstosilicic acid supported catalyst”.

Subsequently, lithium nitrite was weighed to the amount shown in Table 2 and after adding thereto 15 ml of pure water, uniformly dissolved. To this impregnating solution, pure water was added to make the liquid amount shown in Volume of Solution Prepared shown of Table 2 and then the solution was uniformly stirred. The tungstosilicic acid supported catalyst after cooling was added, in the entire amount, to the impregnating solution and impregnated with the solution while thoroughly stirring. The support impregnated with the solution was transferred to a porcelain dish, air-dried for 1 hour and then dried in a hot-air type drier adjusted to 150° C. for 5 hours. After the drying, the catalyst was transferred in a desiccator and left standing to cool to room temperature. The weight of the thus-obtained catalyst was measured.

The result is also shown in Table 2.

TABLE 2

| | | | Weight | | Amount of | | | Volume of | |
|---|---|---|---|---|---|---|---|---|---|
| | | Bulk | of | Kind of | Catalyst | Kind of | Amount of | Solution | Weight |
| Catalyst | | Density | Support | Catalyst | Component | Neutralization | Neutralization | Prepared | after |
| Name | Support | (g/l) | (g) | Component | (g) | Salt | Salt (g) | (ml) | Drying (g) |
| Catalyst 1 | Support 1 | 813 | 81.3 | HSiW | 46.51 | LiNO₃ | 0.0958 | 69 | 121.4 |
| Catalyst 2 | Support 1 | 813 | 81.3 | HSiW | 40.70 | NaOA_{c} | 0.0998 | 69 | 116.8 |
| Catalyst 3 | Support 2 | 456 | 45.6 | HSiW | 40.70 | LiNO₃ | 0.0838 | 43 | 80.8 |
| Catalyst 4 | Support 3 | 575 | 57.5 | HSiW | 34.88 | Cu(NO₃)₂ | 0.3421 | 32 | 87.9 |
| Catalyst 5 | Support 1 | 813 | 81.3 | HPW | 47.51 | LiNO₃ | 0.0917 | 69 | 121.5 |
| Catalyst 6 | Support 4 | 558 | 55.8 | HPW | 65.32 | NaNO₃ | 0.1623 | 34 | 111.3 |
| Catalyst 7 | Support 1 | 813 | 81.3 | HPW | 53.44 | NaNO₃ | 0.1328 | 69 | 126.7 |
| Catalyst 8 | Support 4 | 558 | 55.8 | HSiW | 34.88 | LiNO₃ | 0.1078 | 34 | 86.1 |
| Catalyst 9 | Support 5 | 104.5 | 104.5 | HSiW | 34.88 | Cu(NO₃)₂ | 0.2933 | 31 | 134.2 |

HPW: H₃PW₁₂O₄₀
HSiW: H₄SiW₁₂O₄₀

Preparation Process of Catalyst 2

Support 1 was preliminarily dried for 4 hours in a (hot-air type) drier previously adjusted to 110° C. After the preliminary drying, 1 liter of the support was measured on the bulk density using a 1 liter measuring cylinder. Tungstosilicic acid and sodium acetate each was weighed to the amount shown in Table 2 and after adding thereto 15 ml of pure water, uniformly dissolved to obtain an aqueous solution of Na_{0.1}H_{3.9}SiW₁₂O₄₀ (impregnating solution). To this impregnating solution, pure water was further added to make the liquid amount shown in Volume of Solution Prepared of Table 2. Thereafter, the preliminarily dried support was weighed to the amount shown in Table 2, added to the impregnating solution and impregnated with the solution while thoroughly stirring. The support impregnated with the solution was transferred to a porcelain dish, air-dried for 1 hour and then dried in a hot-air type drier adjusted to 150° C. for 5 hours. After the drying, the catalyst was transferred in a desiccator and left standing to cool to room temperature. The weight of the thus-obtained catalyst was measured.

The result is also shown in Table 2.

Preparation Process of Catalysts 3, 5 and 7

These catalysts were prepared by changing the kind and weight of the support, the kind and weight of the catalyst component, and the weight of the neutralization salt as shown in Table 2 in Preparation Process of Catalyst 1. The loading on a support was performed in the same manner as in Preparation Process of Catalyst 1.

The results are also shown in Table 2.

Preparation Process of Catalysts 4 and 6

These catalysts were prepared by changing the kind and weight of the support, the kind and weight of the catalyst component, and the weight of the neutralization salt as shown in Table 2 in Preparation Process of Catalyst 2. The loading on a support was performed in the same manner as in Preparation Process of Catalyst 2.

The results are also shown in Table 2.

Preparation Process of Catalyst 8

Support 4 was preliminarily dried for 4 hours in a (hot-air type) drier previously adjusted to 110° C. After the preliminary drying, 1 liter of the support was measured on the bulk density using a 1 liter measuring cylinder. Lithium nitrate was weighed to the amount shown in Table 2 and after adding thereto 15 ml of pure water, uniformly dissolved. To this impregnating solution, pure water was further added to make the liquid amount shown in Volume of Solution Prepared of Table 2. Thereafter, the preliminarily dried support was weighed to the amount shown in Table 2, added to the impregnating solution and impregnated with the solution while thoroughly stirring. The support impregnated with the solution was transferred to a porcelain dish, air-dried for 1 hour and then dried in a hot-air type drier adjusted to 150° C. for 5 hours. After the drying, the catalyst was transferred in a desiccator and left standing to cool to room temperature. This is designated as a “lithium nitrate supported catalyst”. Tungstosilicic acid was weighed to the amount shown in Table 2 and after adding thereto 15 ml of pure water, uniformly dissolved.

To this impregnating solution, pure water was added to make the liquid amount shown in Volume of Solution Prepared shown of Table 2 and then the solution was uniformly stirred. The lithium nitrate supported catalyst was added in the entire amount to the impregnating solution and impregnated with the solution while thoroughly stirring. The lithium nitrate supported catalyst impregnated with the solution was transferred to a porcelain dish, air-dried for 1 hour and then dried in a hot-air type drier adjusted to 150° C. for 5 hours. After the drying, the catalyst was transferred in a desiccator and left standing to cool to room temperature. The weight of the thus-obtained catalyst was measured.

The result is also shown in Table 2.

Preparation Process of Catalyst 9

This catalyst was prepared by changing the kind and weight of the support, the kind and weight of the catalyst component, and the weight of the neutralization salt as shown in Table 2 in Preparation Process of Catalyst 8. The loading on a support was performed in the same manner as in Preparation Process of Catalyst 8.

The result is also shown in Table 2.

### EXAMPLE 1

Into a pressure resistant vessel made of SUS 316L, 50 ml of the catalyst obtained in Preparation Process of Catalyst 1 was filled, and then contacted under the contacting conditions shown in Table 3. After the contacting, the vessel was cooled to room temperature and the catalyst used for the contacting (hereinafter referred to as “contacted catalyst”) was drawn out from the vessel. Subsequently, 40 ml of the contacted catalyst was filled into a reaction tube, and a mixed gas of ethylene:acetic acid:steam:nitrogen (=78.5:8.0:4.5:9.0 by volume) was introduced thereinto at a temperature of 165° C., a pressure of 0.8 MPaG (gauge pressure) and a GHSV of 1,500 hr⁻¹, and reacted. The gas passed through the catalyst layer was collected under ice cooling for a predetermined time and the entire amount of condensed components (hereinafter referred to as a “condensed solution”) was recovered and analyzed. The outlet gas remaining uncondensed (hereinafter referred to as an “uncondensed gas”) was measured on the gas flow rate for the same predetermined time as in the condensed solution and then 50 ml of the gas was sampled and analyzed. The results are shown in Table 3.

TABLE 3

| | | Composition of Gas | | | | | Ethyl | Amount of | |
|---|---|---|---|---|---|---|---|---|---|
| | | Treated, acetic acid: | Gas Flow | | Treating | | Acetate, | Butene | Trace By- |
| | | water:nitrogen | Rate | Pressure | Temperature | Treating | STY | Produced | Products |
| | Catalyst | (by mol) | (NL/hr) | (MPaG) | (° C.) | Time (hr) | (g/l-hr) | (vol ppm) | (wt %) |
| Example 1 | Catalyst 1 | 6.0:8.0:86.0 | 60.0 | 0.8 | 160 | 6 | 226 | 213 | 0.0113 |
| Example 2 | Catalyst 1 | $\frac{0.1 : 12.5 : 87.2}{8.0 : 4.5 : 87.5}$ | 60.0 | 0.8 | 160 | $\frac{9}{3}$ | 219 | 292 | 0.0175 |
| Example 3 | Catalyst 1 | 0.0:12.5:87.5 | 60.0 | 0.8 | 160 | 24 | 221 | 113 | 0.0053 |
| Example 4 | Catalyst 2 | 6.0:6.0:88.0 | 60.0 | 0.8 | 165 | 12 | 222 | 625 | 0.0607 |
| Example 5 | Catalyst 2 | 0.0:12.5:87.5 | 60.0 | 0.8 | 165 | 12 | 190 | 3174 | 0.4221 |
| Example 6 | Catalyst 2 | 12.5:0.0:87.5 | 60.0 | 0.8 | 165 | 12 | 219 | 806 | 0.1209 |
| Example 7 | Catalyst 3 | 6.0:6.0:88.0 | 60.0 | 0.8 | 165 | 12 | 273 | 106 | 0.0084 |
| Example 8 | Catalyst 4 | 6.0:6.0:88.0 | 60.0 | 0.8 | 165 | 12 | 215 | 26 | 0.0019 |
| Example 9 | Catalyst 5 | 6.0:6.0:88.0 | 60.0 | 0.8 | 165 | 12 | 228 | 59 | 0.0036 |
| Example 10 | Catalyst 6 | 6.0:6.0:88.0 | 60.0 | 0.8 | 165 | 12 | 203 | 275 | 0.0377 |
| Example 11 | Catalyst 7 | 6.0:6.0:88.0 | 60.0 | 0.8 | 165 | 12 | 240 | 102 | 0.0116 |
| Example 12 | Catalyst 7 | 6.0:6.0:88.0 | 40.0 | 0.8 | 165 | 12 | 235 | 85 | 0.0079 |
| Example 13 | Catalyst 7 | 6.0:6.0:88.0 | 20.0 | 0.8 | 165 | 12 | 230 | 61 | Not |
| | | | | | | | | | detected |
| Example 14 | Catalyst 7 | 6.0:6.0:88.0 | 60.0 | 0.8 | 165 | 1 | 231 | 1224 | 0.0832 |
| Example 15 | Catalyst 8 | 6.0:6.0:88.0 | 60.0 | 0.8 | 165 | 8 | 181 | 200 | 0.0148 |
| Example 16 | Catalyst 9 | 6.0:6.0:88.0 | 60.0 | 0.8 | 165 | 8 | 174 | 189 | 0.0130 |

### EXAMPLES 2 and 3

In the same manner as in Example 1, 50 ml of the catalyst obtained in Preparation Process of Catalyst 1 was filled into a pressure resistant vessel made of SUS 316L and contacted under the contacting conditions shown in Table 3. Thereafter, the catalyst was drawn out, filled into a reaction tube and then subjected to a reaction in the same manner. The results are shown in Table 3.

### EXAMPLE 4

Into a reaction tube, 40 ml of the catalyst obtained in Preparation Process of Catalyst 2 was filled and contacted under the contacting conditions shown in Table 3. Subsequently, a mixed gas of ethylene:acetic acid steam:nitrogen (=78.5:8.0:4.5:9.0 by volume) was introduced thereinto at a temperature of 165° C., a pressure of 0.8 MPaG (gauge pressure) and a GHSV of 1,500 hr⁻¹, and reacted. The reaction results are shown in Table 3.

### EXAMPLES 5 to 16

In the same manner as in Example 4, 40 ml of the catalyst shown in Table 3 was filled into a reaction tube and contacted under the contacting conditions shown in Table 3. Thereafter, a reaction was performed in the same manner as in Example 5. The reaction results are shown in Table 3.

### COMPARATIVE EXAMPLE 1

Into a reaction tube, 40 ml of the same catalyst as in Example 1 was filled and without performing contacting, a mixed gas of ethylene:acetic acid:steamn:nitrogen (=78.5:8.0:4.5:9.0 by volume) was introduced thereinto at a temperature of 165° C., a pressure of 0.8 MPaG (gauge pressure) and a GHSV of 1,500 hr⁻¹, and reacted. The gas passed through the catalyst layer was collected and analyzed in the same manner as in Example 1. The results are shown in Table 4.

TABLE 4

| | | Composition of Gas | | | | | Ethyl | Amount | |
|---|---|---|---|---|---|---|---|---|---|
| | | Treated, acetic acid: | | | Treating | | Acetate, | of Butene | Trace By- |
| | | water: nitrogen | Gas Flow | Pressure | Temperature | Treating | STY | Produced | Products |
| | Catalyst | (by mol) | Rate (Nl/hr) | (MPaG) | (° C.) | Time (hr) | (g/l-hr) | (vol ppm) | (wt %) |
| Comparative | Catalyst 1 | none | — | — | — | — | 210 | 876 | 0.0577 |
| Example 1 | | | | | | | | | |
| Comparative | Catalyst 2 | none | — | — | — | — | 174 | 22593 | 2.8221 |
| Example 2 | | | | | | | | | |
| Comparative | Catalyst 3 | none | — | — | — | — | 268 | 334 | 0.1209 |
| Example 3 | | | | | | | | | |
| Comparative | Catalyst 4 | none | — | — | — | — | 205 | 63 | 0.0162 |
| Example 4 | | | | | | | | | |
| Comparative | Catalyst 5 | none | — | — | — | — | 224 | 207 | 0.0000 |
| Example 5 | | | | | | | | | |
| Comparative | Catalyst 6 | none | — | — | — | — | 191 | 796 | 0.0473 |
| Example 6 | | | | | | | | | |
| Comparative | Catalyst 7 | none | — | — | — | — | 228 | 1585 | 0.1454 |
| Example 7 | | | | | | | | | |
| Comparative | Catalyst 8 | none | — | — | — | — | 174 | 480 | 0.0281 |
| Example 8 | | | | | | | | | |
| Comparative | Catalyst 9 | none | — | — | — | — | 170 | 222 | 0.0294 |
| Example 9 | | | | | | | | | |

### COMPARATIVE EXAMPLES 2 to 9

In the same manner as in Comparative Example 1, 40 ml of the catalyst shown in Table 3 was filled into a reaction tube and a reaction was performed. The reaction results are shown in Table 4.

### Industrial Applicability

It is apparent from the above results that in producing a lower aliphatic carboxylic acid ester from a lower olefin and a lower aliphatic carboxylic acid using a heteropolyacid salt as a catalyst, when the catalyst is contacted with a gas containing at least one member selected from the group consisting of water, lower aliphatic carboxylic acids and lower aliphatic alcohols before performing the reaction, the catalyst obtained can exhibit high initial activity and high space time yield, ensure a sufficiently long catalyst life in the practice in industry, and be greatly prevented from production of by-product compounds harmful to the catalyst, such as butene and aldehydes.

This catalyst can maintain its catalytic activity without performing the removal of by-products, therefore, the catalyst is very useful particularly in practicing the process for producing a lower aliphatic carboxylic acid ester by employing a recycling system.

In the following Examples 17 to 23 and Comparative Examples 10 to 16, the measured values were obtained by analysis or measurement performed according to the following methods.

Analysis Method of Contents of Silicon and Other Elements in Support:

A support was weighed to 1 g and 10 ml of 50% HF (aqueous hydrofluoric acid solution) was added. In this liquid, a sample was dissolved. When an undissolved portion was present, a pressure acidolysis was further performed at 200° C. for 4 hours and thereby, the sample was completely dissolved. This sample solution was appropriately diluted by adding distilled water thereto and quantitated by induction coupled plasma emission spectrometry-mass spectrometry (ICP-MS).

Analysis Method of Silicon Content of Support in Catalyst

This was analyzed according to the above-described analysis method for the silicon content in the support. However, in the case of compounds containing silicon as the heteropolyacid of the catalyst component, such as tungstosilicic acid, the silicon content thereof can be determined by extracting only the heteropolyacid through water extraction and measuring the purity of the support.

Measuring Method of Crush Strength

This was measured according to JIS Z 8841 “Granulate-Strength Test Method”.

Here, the crack ratio of the support was determined as follows. After drying 100 mL of a support at 110° C. for 4 hours, the support was placed in a desiccator and allowed to cool to room temperature. Into a 1 L beaker containing 500 mL of distilled water, 50 mL of the cooled support was charged and after 30 minutes, the proportion of cracked support to non-cracked supports was determined. The obtained value was shown by %.

### EXAMPLES 17 to 24 and COMPARATIVE EXAMPLES 10 to 16

Support:

The following supports were used.

Support 6:

Natural silica (KA-0, produced by SUD-CHEMIE CATALYSTS JAPAN, INC.)

Specific surface area: 103.7 m²/g

Pore volume: 0.33 cm³/g

Crush strength: 62 N

Support 7:

Natural silica (KA-1, produced by SUD-CHEMIE CATALYSTS JAPAN, INC.)

Specific surface area: 110.8 m²/g

Pore volume: 0.42 cm³/g

Crush strength: 84 N

Support 8:

Natural silica (KA-160, produced by SUD-CHEMIE CATALYSTS JAPAN, INC.)

Specific surface area: 158.1 m²/g

Pore volume: 0.42 cm³/g

Crush strength: 71 N

Support 9:

Synthetic silica (N602T, produced by NIKKI CHEMICAL CO., LTD.)

Specific surface area: 132 m²/g

Pore volume: 0.7 cm³/g

Crush strength: 30 N

Support 10:

Synthetic silica (CARIACT Q-10, produced by FUJI SILYSIA CHEMICAL LTD.)

Specific surface area: 309 m²/g

Pore volume: 1.02 cm³/g

Crush strength: 20 N

Support 11:

Synthetic silica (Grace 57, produced by Grace Devison K. K.)

Specific surface area: 430 m²/g

Pore volume: 1.0 cm³/g

Crush strength: 13 N

Support 12:

Silica-alumina (N631L, produced by NIKKI CHIEMICAL CO., LTD.)

Specific surface area: 480 m²/g

Pore volume: 0.18 cm³/g

Crush strength: 1900 N

These supports each was analyzed on the contents of elements. The obtained content as an oxide of each element is shown in Table 5.

Furthermore, the crack ratio of each support is shown in Table 6.

TABLE 5

| | SiO₂ | K₂O | Na₂O | Fe₂O₃ | Al₂O₃ | Others |
|---|---|---|---|---|---|---|
| Support 6 | 96.04 | 0.657 | 1.389 | 0.229 | 0.762 | 0.928 |
| Support 7 | 93.11 | 0.843 | 1.587 | 0.356 | 1.923 | 2.185 |
| Support 8 | 95.60 | 0.778 | 1.046 | 0.132 | 0.698 | 1.750 |
| Support 9 | 98.44 | 0.502 | 0.423 | 0.120 | 0.275 | 0.236 |
| Support 10 | 99.54 | 0.172 | 0.077 | 0.045 | 0.027 | 0.140 |
| Support 11 | 99.71 | 0.105 | 0.105 | 0.049 | 0.032 | 0.002 |
| Support 12 | 81.60 | 0.256 | 0.018 | 0.042 | 12.600 | 5.480 |

Unit: wt %

TABLE 6

| | Crack Ratio % |
|---|---|
| Support 6 | <1.0 |
| Support 7 | <1.0 |
| Support 8 | <1.0 |
| Support 9 | <1.0 |
| Support 10 | 10.0 |
| Support 11 | 15.0 |
| Support 12 | <1.0 |

Production Operation of Catalyst:

Using each of those supports, catalysts were produced as follows.

Supports 6 to 12 were each preliminarily dried for 4 hours in a (hot-air type) drier previously adjusted to 110° C. After the preliminary drying, each support was measured for the bulk density using a 1 liter measuring cylinder. A predetermined amount of tungstosilicic acid was weighed and after adding thereto 15 ml of distilled water, uniformly dissolved. Furthermore, distilled water was added to make an amount described in the column of Prepared Liquid Volume of Table 7. Thereafter, the preliminarily dried support was weighed to a weight described in the column of Weight of Support of Table 7, added to the impregnating solution and impregnated therewith while thoroughly stirring. The support impregnated with the solution was transferred to a porcelain dish, air-dried for 1 hour and then dried in a hot-air type drier adjusted to 150° C. for 5 hours. After the drying, the catalyst was transferred to a desiccator and allowed to cool to room temperature. The thus-obtained catalyst was weighed. Further, the crush strength and crack ratio were measured.

The obtained characteristic data of each catalyst are shown in Table 7.

TABLE 7

| | | Bulk | Weight | | Amount of | Prepared | | | |
|---|---|---|---|---|---|---|---|---|---|
| | | Density of | of | | Catalyst | Liquid | Dry | Crush | Crack |
| Catalyst | Support | Support | Support | | Component | Volume | Weight | Strength | Ratio |
| No. | No. | (g/L) | (g) | Catalyst Comonent | Supported (g/L) | (ml) | (g) | (N) | (%) |
| 10 | 6 | 558 | 55.8 | Li_{0.01}H_{3.99}SiW₁₂O₄₀ | 300 | 34 | 86.2 | 60 | <1.0 |
| 11 | 6 | 558 | 55.8 | Li_{0.1}H_{2.9}PW₁₂O₄₀ | 550 | 34 | 110.5 | 66 | <1.0 |
| 12 | 7 | 573 | 57.3 | Li_{0.01}H_{3.99}SiW₁₂O₄₀ | 300 | 33 | 87.1 | 81 | <1.0 |
| 13 | 8 | 575 | 57.5 | H₄SiW₁₂O₄₀ | 300 | 33 | 87.9 | 70 | <1.0 |
| 14 | 8 | 575 | 57.5 | Li_{0.001}H_{2.999}PW₁₂O₄₀ | 300 | 34 | 88.1 | 69 | <1.0 |
| 15 | 9 | 448 | 44.8 | Li_{0.1}H_{2.9}PW₁₂O₄₀ | 400 | 41 | 85.2 | 28 | <1.0 |
| 16 | 10 | 456 | 45.6 | Li_{0.1}H_{3.9}SiW₁₂O₄₀ | 300 | 43 | 75.9 | 16 | 8.2 |
| 17 | 10 | 456 | 45.6 | H₃PW₁₂O₄₀ | 350 | 43 | 80.9 | 17 | 8.9 |
| 18 | 11 | 413 | 41.3 | Li_{0.1}H_{3.9}SiW₁₂O₄₀ | 300 | 50 | 71.0 | 12 | 14.2 |
| 19 | 11 | 413 | 41.3 | Li_{0.1}H_{3.9}SiW₁₂O₄₀ | 350 | 50 | 76.2 | 11 | 15.6 |
| 20 | 12 | 607 | 60.7 | H₃PW₁₂O₄₀ | 300 | 33 | 90.5 | 1800 | <1.0 |

Production Operation of Carboxylic Acid Ester

Into a pressure-resistant vessel made of SUS316L, 40 ml of each catalyst obtained above was filled, and a reaction was carried out under the conditions in the column of Pre-Treatment Conditions and the column of Ethyl Acetate Production Conditions. The gas passed through the catalyst layer was cooled and collected under ice cooling for a predetermined time and the entire amount was recovered (this is called a condensed solution) and analyzed. The outlet gas remaining uncondensed (this is called an uncondensed gas) was measured on the gas flow rate for the same predetermined time as in the condensed solution and then 50 ml of the uncondensed gas was sampled and analyzed.

The results obtained are shown in Tables 8 and 9.

TABLE 8

| | | | | | | | Ethyl Acetate Production Conditions | | | | Reaction Results | | |
|---|---|---|---|---|---|---|---|---|---|---|---|---|---|
| | | Pre-Treatment Condition | | | | | Composition of | | | | | Ethyl | |
| | | Treated Gas | Gas | | | | Starting Gases | | | Tem- | | Acetate | Selectivity |
| Exam- | Cata- | Composition | Flow | | Temper- | | C₂H₄:HOAc: | Gas Flow | | per- | Reaction | STY | of |
| ple | lyst | HOAc:H₂O | Rate | Pressure | ature | Time | H₂O:N₂ | Rate | Pressure | ature | Time | (g/L · | Butene |
| No. | No. | (molar ratio) | (NL/hr) | (MPaG) | (° C.) | (hr) | (molar ratio) | (NL/hr) | (MPaG) | (° C.) | (hr) | hr) | (%) |
| 17 | 10 | none | — | — | — | — | 78.5:8:4.5:9 | 60 | 0.8 | 165 | 5 | 181 | 0.01 |
| 18 | 11 | none | — | — | — | — | 70:8:4:18 | 60 | 0.6 | 165 | 5 | 186 | 1.97 |
| 19 | 12 | none | — | — | — | — | 70:8:4:18 | 40 | 1.0 | 165 | 5 | 184 | 0.21 |
| 20 | 13 | none | — | — | — | — | 70:8:4:18 | 80 | 0.8 | 165 | 5 | 187 | 1.19 |
| 21 | 14 | none | — | — | — | — | 78.5:8:4.5:9 | 60 | 0.6 | 165 | 5 | 174 | 1.27 |
| 22 | 15 | none | — | — | — | — | 70:8:3:19 | 60 | 0.8 | 170 | 5 | 190 | 2.34 |
| 23 | 11 | 6:6 | 60 | 0.8 | 165 | 12 | 78.5:8:4.5:9 | 60 | 0.8 | 165 | 5 | 208 | 0.22 |
| 24 | 14 | 6:6 | 60 | 0 | 165 | 12 | 78.5:8:4.5:9 | 60 | 0.8 | 165 | 5 | 205 | 0.26 |

TABLE 9

| Com- | | | | | | | Ethyl Acetate Production Conditions | | | | Reaction Results | | |
|---|---|---|---|---|---|---|---|---|---|---|---|---|---|
| par- | | Pre-Treatment Condition | | | | | Composition of | | | | | Ethyl | |
| ative | | Treated Gas | Gas | | | | Starting Gases | | | Tem- | | Acetate | Selectivity |
| Exam- | Cata- | Composition | Flow | | Temper- | | C₂H₄:HOAc: | Gas Flow | | per- | Reaction | STY | of |
| ple | lyst | HOAc:H₂O | Rate | Pressure | ature | Time | H₂O:N₂ | Rate | Pressure | ature | Time | (g/L · | Butene |
| No. | No. | (molar ratio) | (NL/hr) | (MPaG) | (° C.) | (hr) | (molar ratio) | (NL/hr) | (MPaG) | (° C.) | (hr) | hr) | (%) |
| 10 | 16 | none | — | — | — | — | 78.5:8:4.5:9 | 60 | 0.8 | 165 | 5 | 254 | 0.22 |
| 11 | 17 | none | — | — | — | — | 70:8:4:18 | 60 | 0.6 | 165 | 5 | 250 | 0.14 |
| 12 | 18 | none | — | — | — | — | 70:8:4:18 | 40 | 1.0 | 165 | 5 | 244 | 0.12 |
| 13 | 19 | none | — | — | — | — | 70:8:4:18 | 80 | 0.8 | 165 | 5 | 260 | 0.13 |
| 14 | 20 | none | — | — | — | — | 78.5:8:4.5:9 | 60 | 0.6 | 165 | 5 | 12 | 0.00 |
| 15 | 17 | 6:6 | 60 | 0.8 | 165 | 12 | 78.5:8:4.5:9 | 60 | 0.8 | 165 | 5 | 254 | 0.12 |
| 16 | 19 | 6:6 | 60 | 0 | 165 | 12 | 78.5:8:4.5:9 | 60 | 0.8 | 165 | 5 | 273 | 0.08 |

Analysis Method for Condensed Solution:

An analysis solution was prepared by adding 1 ml of 1,4-dioxane as the internal standard to 10 ml of the reaction solution, 0.4 μl of the analysis solution was injected, and the analysis was performed using the internal standard method under the following conditions.

Gas Chromatography:

GC-14B, Manufactured by Shimadzu Corporation Column:

capillary column TC-WAX (length: 30 m, internal diameter: 0.25 mm, film thickness: 0.25 μm)

Carrier Gas:

nitrogen (split ratio: 20, column flow rate: 1 ml/min)

Temperature Conditions:

The detector and the vaporization chamber were at a temperature of 200° C. and the column temperature was kept at 40° C. for 7 minutes from the initiation of analysis, thereafter elevated up to 230° C. at a temperature rising rate of 10° C./min, and kept at 230° C. for 5 minutes.

Detector:

FID (H₂ pressure: 70 KPa, air pressure: 100 KPa)

Analysis Method for Uncondensed Gas:

Using an absolute calibration curve method, the analysis was performed under the following conditions by sampling 50 ml of the effluent gas and passing the whole amount thereof into a 1 ml-volume gas sampler attached to the gas chromatograph.

1. Ether, Carboxylic Acid Ester, Alcohol and Trace by-Products

Gas Chromatograph:

gas chromatograph (GC-14, manufactured by Shimadzu Corporation) with a gas sampler for Shimadzu gas chromatograph (MGS-4, measuring tube: 1 ml)

Column:

packed column SPAN 80, 15% Shinchrom A of 60 to 80 mesh (length: 5 m)

Carrier Gas:

nitrogen (flow rate: 25 ml/min)

Temperature Conditions:

the detector and the vaporization chamber were at a temperature of 120° C., and the column temperature was 65° C. and constant.

Detector:

FID (H₂ pressure: 60 KPa, air pressure: 100 KPa)

2. Butene:

Gas Chromatograph:

gas chromatograph (GC-14B, manufactured by Shimadzu Corporation) with a gas sampler for Shimadzu gas chromatograph (MGS-4, measuring tube: 1 ml)

Column:

packed column Unicarbon A-400 of 80/100 mesh, length: 2 m

Carrier Gas:

helium (flow rate: 23 ml/min)

Temperature Conditions:

the detector and the vaporization chamber were at a temperature of 130° C., and the column temperature was elevated from 40° C. to 95° C. at a temperature rising rate of 40°/min.

Detector:

FID (H₂ pressure: 70 KPa, air pressure: 100 KPa)

3. Ethylene:

Gas Chromatograph:

gas chromatograph (GC-14B, manufactured by Shimadzu Corporation) with a gas sampler for Shimadzu gas chromatograph (MGS-4, measuring tube: 1 ml)

Column:

packed column Unibeads IS, length: 3 m

Carrier Gas:

helium (flow rate: 20 ml/min)

Temperature Conditions:

the detector and the vaporization chamber were at a temperature of 120° C., and the column temperature was 65° C. and constant.

Detector:

TCD (He pressure: 70 KPa, current: 90 mA, temperature: 120° C.)

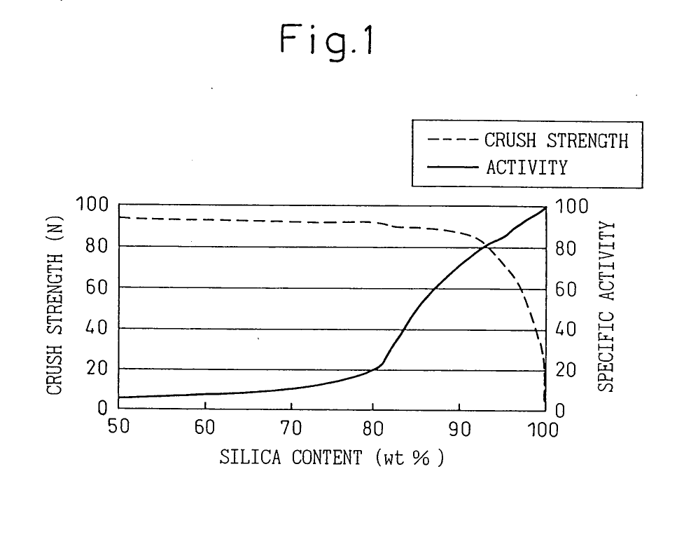

FIG. 1 is a graph where based on the results above, the crush strength (N) of the catalyst support and the specific activity of the catalyst are plotted with respect to the silica content (wt %). It is seen from FIG. 1 that when the silicon content of the siliceous support according to the present invention is in the range of 85 to 99% by mass in terms of silica, the obtained catalyst can have high strength and high activity.

It is apparent from the above results that according to the present invention, at the time of producing a lower aliphatic carboxylic acid ester by reacting a lower olefin with a lower aliphatic monocarboxylic acid in a gas phase, a catalyst having a silicon content in a predetermined range is used as the siliceous support of the catalyst for the production, whereby a catalyst having predetermined strength and exhibiting performances of a predetermined level can be obtained and the production operation can be stably performed without causing cracking or abrasion of the catalyst even in long-term use.

What is claimed is:

1. A catalyst for use in producing a lower aliphatic carboxylic acid ester, which is used in reacting a lower olefin with a lower aliphatic carboxylic acid in a gas phase to produce a lower aliphatic carboxylic acid ester, wherein the catalyst is produced by a process comprising the following first and second steps:
First Step
a step for loading one or more heteropolyacid salts on a support to obtain a heteropolyacid salt supported catalyst; and
Second step
a step for contacting the heteropolyacid salt supported catalyst obtained in the first step with a gas containing at least one member selected from the group consisting of water, lower aliphatic carboxylic acids and lower aliphatic alcohols to obtain the catalyst for use in producing a lower aliphatic carboxylic acid ester.

2. The catalyst as claimed in claim 1, wherein the second step is performed in the reactor used in reacting the lower olefin with the lower aliphatic carboxylic acid in a gas phase.

3. The catalyst as claimed in claim 1 or 2, wherein the heteropolyacid salt is at least one salt selected from the group consisting of lithium salts, sodium salts, magnesium salts, barium salts, copper salts, gold salts and gallium salts of silicotungstic acid, phosphotungstic acid, phosphomolybdic acid, silicomolybdic acid, silicovanadotungstic acid, phosphovanadotungstic acid and phosphovanadomolybdic acid.

4. A process for producing a catalyst for use in producing a lower aliphatic carboxylic acid ester, said catalyst being used in reacting a lower olefin with a lower aliphatic carboxylic acid in a gas phase to produce a lower aliphatic carboxylic acid ester, which process comprises the following first and second steps:
First Step
a step for loading one or more heteropolyacid salts on a support to obtain a heteropolyacid salt supported catalyst; and
Second Step
a step for contacting the heteropolyacid salt supported catalyst obtained in the first step with a gas containing at least one member selected from the group consisting of water, lower aliphatic carboxylic acids and lower aliphatic alcohols to obtain the catalyst for use in producing a lower aliphatic carboxylic acid ester.

5. The process as claimed in claim 4, wherein the second step is performed in the reactor used in reacting the lower olefin with the lower aliphatic carboxylic acid in a gas phase.

6. The process as claimed in claim 4 or 5, wherein the heteropolyacid salt is at least one salt selected from the group consisting of lithium salts, sodium salts, magnesium salts, barium salts, copper salts, gold salts and gallium salts of silicotungstic acid, phosphotungstic acid, phosphomolybdic acid, silicomolybdic acid, silicovanadotungstic acid, phosphovanadotungstic acid and phosphovanadomolybdic acid.

7. The process as claimed in any one of claims 4 or 5, wherein the second step is performed at a temperature of 80 to 300° C.

8. The process as claimed in any one of claims 4 or 5, wherein the second step is performed under a pressure of 0 to 3 MPaG (gauge pressure).

9. The process as claimed in any one of claims 4 or 5, wherein the second step is performed at a gas hourly space velocity (GHSV) of 100 to 7,000 hr⁻¹.

10. The process as claimed in any one of claims 4 or 5, wherein the gas containing at least one member selected from the group consisting of water, lower aliphatic carboxylic acids and lower aliphatic alcohols used in the second step is a mixed gas of water and acetic acid.

11. A process for producing a lower aliphatic carboxylic acid ester, comprising reacting a lower olefin with a lower aliphatic carboxylic acid in a gas phase in the presence of the catalyst as claimed in any one of claims 1 or 2.

12. A process for producing a lower aliphatic carboxylic acid ester, comprising reacting a lower olefin with a lower aliphatic carboxylic acid in a gas phase in the presence of water and the catalyst as claimed in any one of claims 1 or 2.

13. A process for producing a lower aliphatic carboxylic acid ester, comprising reacting a lower olefin with a lower aliphatic carboxylic acid in a gas phase, which process comprises the following first to fourth steps:
First Step
a step of loading one or more heteropolyacid salts on a support to obtain a heteropolyacid salt supported catalyst;
Second Step
a step of filling the heteropolyacid salt supported catalyst obtained in the first step, into a reactor for use in the reaction of the lower olefin with the lower aliphatic carboxylic acid in a gas phase;
Third Step
a step of contacting the heteropolyacid salt supported catalyst filled in the reactor, with a gas containing at least one member selected from the group consisting of water, lower aliphatic carboxylic acids and lower aliphatic alcohols; and
Fourth Step
a step of passing a mixed gas containing the lower olefin and the lower aliphatic carboxylic acid through the heteropolyacid salt supported catalyst after the third step, to obtain the lower aliphatic carboxylic acid ester.

14. The process as claimed in claim 13, wherein the heteropolyacid salt is at least one salt selected from the group consisting of lithium salts, sodium salts, magnesium salts, barium salts, copper salts, gold salts and gallium salts of silicotungstic acid, phosphotungstic acid, phosphomolybdic acid, silicomolybdic acid, silicovanadotungstic acid, phosphovanadotungstic acid and phosphovanadomolybdic acid.

15. The process as claimed in claim 13 or 14, wherein the third step is performed at a temperature of 80 to 300° C.

16. The process as claimed in any one of claims 13 or 14, wherein the third step is performed under a pressure of 0 to 3 MPaG (gauge pressure).

17. The process as claimed in any one of claims 13 or 14, wherein the third step is performed at a gaseous hourly space velocity (GHSV) of 100 to 7,000 hr⁻¹.

18. The process as claimed in any one of claims 13 or 14, wherein the lower aliphatic carboxylic acid used in the third step is the same as the lower aliphatic carboxylic acid used in producing the lower aliphatic carboxylic acid ester.

19. The process as claimed in any one of claims 13 or 14, wherein the mixed gas containing a lower olefin and a lower aliphatic carboxylic acid used in the fourth step contains water.

20. A catalyst for use in producing a lower aliphatic carboxylic acid ester, which is used in reacting a lower olefin with a lower aliphatic carboxylic acid in a gas phase to produce a lower aliphatic carboxylic acid ester, wherein the catalyst is produced by a process comprising the following first and second steps:
First Step
a step of loading one or more heteropolyacid salts on a support to obtain a heteroolyacid salt supported catalyst; and
Second Step
a step of contacting the heteropolyacid salt supported catalyst obtained in the first step with a gas containing at least one member selected from the group consisting of water, lower aliphatic carboxylic acids and lower aliphatic alcohols, in the absence of any lower olefin, to obtain the catalyst for use in producing a lower aliphatic carboxylic acid ester;
wherein the heteropolyacid salt is at least one salt selected from the group consisting of lithium salts, sodium salts, magnesium salts, barium salts, copper salts, gold sats and gallium salts of silicotungstic acid, phosphotungstic acid, phosphomolybdic acid, silicomolybdic acid, silicovanadotungstic acid, phosphovanadotungstic acid and phosphovanadomolybdic acid.

21. A process for producing a catalyst for use in producing a lower aliphatic carboxylic acid ester, said catalyst being used in reacting a lower olefin with a lower aliphatic carboxylic acid in a gas phase to produce a lower aliphatic carboxylic acid ester, which process comprises the following first and second steps:
First Step
a step of loading one or more heteropolyacid salts on a support to obtain a heteropolyacid salt supported catalyst; and
Second Step
a step of contacting the heteropolyacid salt supported catalyst obtained in the first step with a gas containing at least one member selected from the group consisting of water, lower aliphatic carboxylic acids and lower aliphatic alcohols, in the absence of any lower olefin, to obtain the catalyst for use in producing a lower aliphatic carboxylic acid ester;
wherein the heteropolyacid salt is at least one salt selected from the group consisting of lithium salts, sodium salts, magnesium salts, barium salts, copper salts, gold salts and gallium salts of silicotungstic acid, phosphotungstic acid, phosphomolybdic acid, silicomolybdic acid, silicovanadotungstic acid, phosphovanadotungstic acid and phosphovanadomolybdic acid.

22. A process for producing a lower aliphatic carboxylic acid ester, comprising reacting a lower olefin with a lower aliphatic carboxylic acid in a gas phase, which process comprises the following first to fourth steps:
First Step
a step of loading one or more heteropolyacid salts on a support to obtain a heteropolyacid salt supported catalyst;
Second Step
a step of filling the heteropolyacid salt supported catalyst obtained in the first step, into a reactor for use in the reaction of the lower olefin with the lower aliphatic carboxylic acid in a gas phase;
Third Step
a step of contacting the heteropolyacid salt supported catalyst filled in the reactor, with a gas containing at least one member selected from the group consisting of water, lower aliphatic carboxylic acids and lower aliphatic alcohols, in the absence of any lower olefin, and
Fourth Step
a step of passing a mixed gas containing the lower olefin and the lower aliphatic carboxylic acid through the heteropolyacid salt supported catalyst after the third step, to obtain the lower aliphatic carboxylic acid ester;
wherein the heteropolyacid salt is at least one salt selected from the group consisting of lithium salts, sodium salts, magnesium salts, barium salts, copper salts, gold salts and gallium salts of silicotungstic acid, phosphotungstic acid, phosphomolybdic acid, phosphovanadotungstic acid and phosphovanadomolybdic acid.

23. A siliceous support for use in a catalyst for producing a lower aliphatic carboxylic acid ester by reacting a lower olefin with a lower aliphatic carboxylic acid in a gas phase, which has a silicon content of from 39.7 to 46.3% by mass.

24. A siliceous support for use in a catalyst for producing a lower aliphatic carboxylic acid ester by reacting a lower olefin with a lower aliphatic carboxylic acid in a gas phase, which has a silicon content of from 85 to 99% by mass in terms of silicon dioxide.

25. A siliceous support for use in a catalyst for producing a lower aliphatic carboxylic acid ester by reacting a lower olefin with a lower aliphatic carboxylic acid in a gas phase, which has a crush strength of 30 N or more.

26. A catalyst for producing a lower aliphatic carboxylic acid ester by reacting a lower olefin with a lower aliphatic carboxylic acid in a gas phase, comprising a support as claimed in any one of claims 23 to 25.

27. A catalyst according to claim 26, which has a crush strength of 30 N or more.

28. A catalyst according to claim 26, wherein at least one member selected from the group consisting of heteropolyacids and salts thereof is supported on the support.

29. A catalyst according to claim 28, wherein the heteropolyacids are selected from the group consisting of tungstosilisic acid, tungstophosphoric acid, molybdophosphoric acid, molybdosilisic acid, vanadotungstosilisic acid, vanadotungstophosphoric acid, vanadomolybdophosphoric acid, vanadomolybdosilisic acid, molybdotungstosilisic acid and molybdotungstophosphoric acid.

30. A catalyst according to claim 28, wherein the heteropolyacid salts are selected from the group consisting of lithium, sodium, magnesium, barium, copper, gold and gallium salts of tungstosilisic acid, tungstophosphoric acid, molybdophosphoric acid, molybdosilisic acid, vanadotungstosilisic acid, vanadotungstophosphoric acid, vanadomolybdophosphoric acid, vanadomolybdosilisic acid, molybdotungstosilisic acid and molybdotungstophosphoric acid.

31. A process for producing a catalyst comprising loading at least one member selected from the group consisting of heteropolyacids and salts thereof on a support as claimed in any one of claims 23 to 25.

32. A process for producing a catalyst comprising the following first and second steps:
First Step:
a step of loading at least one member selected from the group consisting of heteropolyacids and salts thereof on a support as claimed in any one of claims 23 to 25 to obtain a heteropolyacid and/or heteropolyacid salt-supported catalyst; and
Second Step:
a step of contacting the heteropolyacid and/or heteropolyacid salt-supported catalyst obtained in the first step with a gas containing at least one member selected from the group consisting of water, lower aliphatic carboxylic acids and lower aliphatic alcohols to obtain a catalyst for producing a lower aliphatic carboxylic acid ester.

33. A process according to claim 32, wherein the gas containing at least one member selected from the group consisting of water, lower aliphatic carboxylic acids and lower aliphatic alcohols is a mixed gas of water and acetic acid.

34. A process for producing a lower aliphatic carboxylic acid ester, comprising reacting a lower olefin with a lower aliphatic carboxylic acid in a gas phase in the presence of a catalyst as claimed in claim 26.

35. A process according to claim 34, wherein the reaction of a lower olefin with a lower aliphatic carboxylic acid is carried out in the presence of water.

36. A process for producing a catalyst comprising the following first and second steps:
First Step:
a step of loading at least one member selected from the group consisting of heteropolyacids and salts thereof on a support as claimed in any one of claims 23 to 25 to obtain a heteropolyacid and/or heteropolyacid salt-supported catalyst; and
Second Step:
a step of contacting the heteropolyacid and/or heteropolyacid salt-supported catalyst obtained in the first step with a gas containing at least one member selected from the group consisting of water, lower aliphatic carboxylic acids and lower aliphatic alcohols, in the absence of any lower olefin, to obtain a catalyst for producing a lower aliphatic carboxylic acid ester.
